(19) United States
(12) Patent Application Publication
(10) Pub. No.: US 2024/0013928 A1
(43) Pub. Date: Jan. 11, 2024

(54) SYSTEMS AND METHODS OF PATIENT PRIORITIZATION SCORES AND MEASURES

(71) Applicant: Persivia Inc., Marlborough, MA (US)

(72) Inventors: Mansoor Khan, Shrewsbury, MA (US); Fauzia Khan, Shrewsbury, MA (US)

(73) Assignee: Persivia Inc., Marlborough, MA (US)

(21) Appl. No.: 18/349,952

(22) Filed: Jul. 10, 2023

Related U.S. Application Data
(60) Provisional application No. 63/388,197, filed on Jul. 11, 2022.

(51) Int. Cl. G16H 50/70; G16H 40/20; G16H 50/30; G06F 40/40

(52) U.S. Cl. CPC G16H 50/70; G16H 40/20; G16H 50/30; G06F 40/40

(57) ABSTRACT

Systems and method for determining patient prioritization scores for use by healthcare professionals in administering care to patients. Healthcare data of patients is used to determine one or more of a care gap score representing opportunities to improve care of a patient, a risk score representing risks to health of the patient, a change over time in the risk score for the patient, a measure representing a willingness of the patient to receive care, and a prediction of future costs associated with care of the patient. The one or more scores and measures are used to determine a patient prioritization score for the patient. A healthcare professional can be prompted based on the patient prioritization to administer of care to the patient. A graphical user interface (GUI) can be generated that includes the prompt, and the GUI may be provided to a display.

### CROSS REFERENCE TO RELATED APPLICATIONS

This application claims the benefit of priority under 35 U.S.C. § 119(e) to U.S. Provisional Application Ser. No. 63/388,197, titled “SYSTEMS AND METHODS OF PATIENT PRIORITIZATION SCORES AND MEASURES,” and filed on Jul. 11, 2022, the entire contents of which are incorporated by reference herein.

### BACKGROUND

Healthcare providers may have limited resources and limited time to impact care of patients.

### SUMMARY

According to aspects of the disclosure, there is provided a method of determining a patient prioritization score of each particular patient of a plurality of patients for use by a healthcare professional in administering care to the plurality of patients, the method comprising: receiving healthcare data for each particular patient of the plurality of patients; determining, based on the healthcare data for each particular patient, a respective patient prioritization score for each particular patient of the plurality of patients, the determining comprising: determining a care gap score for the particular patient based on a first subset of the healthcare data associated with the particular patient, the care gap score representing opportunities to improve care of the patient; determining a risk score for the particular patient based on a second subset of the healthcare data associated with the particular patient, the risk score representing risks to health of the patient; determining a change over a time period in the risk score for the particular patient; determining a measure representing a willingness of the particular patient to receive care; determining a prediction of future costs associated with care of the particular patient; determining the patient prioritization score for the particular patient based on the care gap score, the risk score, the change over the time period, and the prediction of the future costs; determining, based on the respective patient prioritization score determined for each of the plurality of patients, a respective level of care to be administered to each particular patient of the plurality of patients; and prompting the healthcare professional to administer the determined respective level of care to each particular patient of the plurality of patients.

Optionally, the plurality of patients includes at least 2 patients, at least 5 patients, at least patients, at least 25 patients, at least 50 patients, at least 100 patients, at least 500 patients, at least 1,000 patients, or at least 5,000 patients.

Optionally, determining the respective level of care to be administered to each particular patient of the plurality of patients comprises: determining that more resources should be allocated to patients of the plurality of patients for whom a higher patient prioritization score was determined than patients for whom a lower patient prioritization score was determined.

Optionally, determining a care gap score for the particular patient based on the first subset of healthcare data for the particular patient comprises: applying a rule to the healthcare data for the particular patient, wherein the rule is associated with a care gap; determining, based on a result of applying the rule, whether the particular patient has the care gap associated with the rule; providing an individual score for the care gap based on a result of determining whether the particular patient has the care gap; applying a weight to the individual score for the care gap; and determining the care gap score based on the weight and the individual score.

Optionally, the first subset of the healthcare data associated with the particular patient comprises at least one of demographics, diagnosis, social history, family history, medical visit data, radiology, procedures, lab values, lab trends, lab deltas, medication, follow ups, genetic markers, exercise habits, and shopping habits; and applying the rule to the first subset of healthcare data for the particular patient comprises applying the rule to the at least one of demographics, diagnosis, social history, family history, medical visit data, radiology, procedures, lab values, lab trends, lab deltas, medication, follow up, genetic markers, exercise habits, and shopping habits.

Optionally, receiving the healthcare data for each patient of the plurality of patients comprises: using a large language model to generate a respective prompt requesting input from each particular patient of the plurality of patients; and receiving at least some of the healthcare data in response to generating the respective prompt requesting input from each particular patient of the plurality of patients.

According to aspects of the disclosure, there is provided a method of determining a patient prioritization score of a patient for use by a healthcare professional in administering care to the patient. The method comprises receiving healthcare data associated with the patient, determining, based on the healthcare data for the patient, a care gap score for the patient representing opportunities to improve care of the patient, determining, based on the healthcare data for the patient, a risk score for the patient representing risks to health of the patient, determining, based on the care gap score and the risk score, the patient prioritization score of the patient, prompting, based on the patient prioritization score for the patient, the healthcare professional to administer a level of care to the patient.

Optionally, determining a care gap score for the patient based on the healthcare data for the patient comprises applying a rule to the healthcare data for the patient, wherein the rule is associated with a care gap, determining, based on a result of applying the rule, whether the patient has the care gap associated with the rule, providing an individual score for the care gap based on a result of determining whether the patient has the care gap, applying a weight to the individual score for the care gap, and determining the care gap score based on the weight and the individual score.

Optionally, the healthcare data associated with the patient comprises at least one of demographics, diagnosis, social history, family history, medical visit data, radiology, procedures, lab values, lab trends, lab deltas, medication, follow ups, genetic markers, exercise habits, and shopping habits and applying the rule to the healthcare data for the patient comprises applying the rule to the at least one of demographics, diagnosis, social history, family history, medical visit data, radiology, procedures, lab values, lab trends, lab deltas, medication, follow up, genetic markers, exercise habits, and shopping habits.

Optionally, the healthcare data associated with the patient comprises at least one of clinical data, claims data, event data, device data, patient reported data or socio-economic data, and determining a risk score for the patient based on the healthcare data for the patient comprises determining the risk score for the patient based on the at least one of the clinical data, claims data, event data, device data, patient reported data or socio-economic data.

Optionally, the method further comprises determining a change over a time period in the risk score for the patient and determining the patient prioritization score further based on the change over the time period in the risk score for the patient.

Optionally, the method further comprises determining a measure representing a willingness of the patient to receive care and determining the patient prioritization score further based on the measure representing the willingness of the patient to receive care.

Optionally, the method further comprises determining a prediction of future costs associated with care of the patient and determining the patient prioritization score further based on the prediction of the future costs associated with care of the patient.

Optionally, receiving the healthcare data associated with the patient comprises: using a large language model to generate a prompt requesting input from the patient; and receiving at least some of the healthcare data in response to generating the prompt.

According to aspects of the disclosure, there is provided at least one non-transitory computer-readable storage medium having instructions encoded thereon that, when executed by at least one computer processor, cause the at least one computer processor to perform a method of determining a patient prioritization score for a patient. The method comprises receiving healthcare data associated with the patient, determining, based on the healthcare data for the patient, a care gap score for the patient representing opportunities to improve care of the patient, determining, based on the healthcare data for the patient, a risk score for the patient representing risks to health of the patient, determining, based on the care gap score and the risk score, the patient prioritization score of the patient, prompting, based on the patient prioritization score for the patient, the healthcare professional to administer a level of care to the patient.

Optionally, determining a care gap score for the patient based on the healthcare data for the patient comprises applying a rule to the healthcare data for the patient, wherein the rule is associated with a care gap, determining, based on a result of applying the rule, whether the patient has the care gap associated with the rule, providing an individual score for the care gap based on a result of determining whether the patient has the care gap, applying a weight to the individual score for the care gap, and determining the care gap score based on the weight and the individual score.

Optionally, the healthcare data associated with the patient comprises at least one of demographics, diagnosis, social history, family history, medical visit data, radiology, procedures, lab values, lab trends, lab deltas, medication, follow ups, genetic markers, exercise habits, and shopping habits and applying the rule to the healthcare data for the patient comprises applying the rule to the at least one of demographics, diagnosis, social history, family history, medical visit data, radiology, procedures, lab values, lab trends, lab deltas, medication, follow up, genetic markers, exercise habits, and shopping habits.

Optionally, the healthcare data associated with the patient comprises at least one of clinical data, claims data, event data, device data, patient reported data or socio-economic data and determining a risk score for the patient based on the healthcare data for the patient comprises determining the risk score for the patient based on the at least one of the clinical data, claims data, event data, device data, patient reported data or socio-economic data.

Optionally, the method further comprises determining a change over a time period in the risk score for the patient and determining the patient prioritization score further based on the change over the time period in the risk score for the patient.

Optionally, the method further comprises determining a measure representing a willingness of the patient to receive care and determining the patient prioritization score further based on the measure representing the willingness of the patient to receive care.

Optionally, the method further comprises determining a prediction of future costs associated with care of the patient and determining the patient prioritization score further based on the prediction of the future costs associated with care of the patient.

According to aspects of the disclosure, there is provided at least one non-transitory computer-readable storage medium having instructions encoded thereon that, when executed by at least one computer processor, cause the at least one computer processor to perform a method. The method comprises generating a graphical user interface (GUI) including a prompt to a healthcare professional to administer a level of care to a patient and providing the GUI to a display. The prompt, included in the GUI, to the healthcare professional to administer the level of care to the patient is determined based on a patient prioritization score of the patient, the patient prioritization score of the patient being determined based on a care gap score for the patient representing opportunities to improve care of the patient and a risk score for the patient representing risks to health of the patient.

Optionally, the method further comprises receiving healthcare data associated with the patient, determining, based on the healthcare data for the patient, the care gap score for the patient representing opportunities to improve care of the patient, determining, based on the healthcare data for the patient, the risk score for the patient representing risks to health of the patient, and determining, based on the care gap score and the risk score, the patient prioritization score of the patient.

Optionally, determining a care gap score for the patient based on the healthcare data for the patient comprises applying a rule to the healthcare data for the patient, wherein the rule is associated with a care gap, determining, based on a result of applying the rule, whether the patient has the care gap associated with the rule, providing an individual score for the care gap based on a result of determining whether the patient has the care gap, applying a weight to the individual score for the care gap, and determining the care gap score based on the weight and the individual score.

Optionally, the healthcare data associated with the patient comprises at least one of demographics, diagnosis, social history, family history, medical visit data, radiology, procedures, lab values, lab trends, lab deltas, medication, follow ups, genetic markers, exercise habits, and shopping habits and the rule to the healthcare data for the patient comprises applying the rule to the at least one of demographics, diagnosis, social history, family history, medical visit data, radiology, procedures, lab values, lab trends, lab deltas, medication, follow up, genetic markers, exercise habits, and shopping habits.

Optionally, the healthcare data associated with the patient comprises at least one of clinical data, claims data, event data, device data, patient reported data or socio-economic data and determining a risk score for the patient based on the healthcare data for the patient comprises determining the risk score for the patient based on the at least one of the clinical data, claims data, event data, device data, patient reported data or socio-economic data.

Optionally, the method further comprises determining a change over a time period in the risk score for the patient and determining the patient prioritization score further based on the change over the time period in the risk score for the patient.

Optionally, the method further comprises determining a measure representing a willingness of the patient to receive care and determining the patient prioritization score further based on the measure representing the willingness of the patient to receive care.

Optionally, the method further comprises determining a prediction of future costs associated with care of the patient and determining the patient prioritization score further based on the prediction of the future costs associated with care of the patient.

### BRIEF DESCRIPTION OF DRAWINGS

Various aspects and embodiments will be described with reference to the following figures. The figures are not necessarily drawn to scale.

FIG. 1A is a diagram depicting an illustrative technique 100 for determining a patient prioritization score of a patient, according to embodiments of the technology described herein.

FIG. 1B is a diagram depicting an illustrative technique 150 for determining the patient prioritization score shown in FIG. 1A, according to embodiments of the technology described herein.

FIG. 2 is a block diagram of an example system 200 for determining a patient prioritization score for a patient, according to embodiments of the technology described herein.

FIG. 3A is a flowchart of an illustrative process for determining a patient prioritization score for a patient, according to embodiments of the technology described herein.

FIG. 3B shows an example table for determining a patient prioritization score (PPS) based on one or more factors, according to embodiments of the technology described herein.

FIG. 3C shows an example of determining a PPS for a patient using the example table shown in FIG. 3B.

FIG. 4 is a flowchart of an illustrative process for determining a care gap score for a patient, according to embodiments of the technology described herein.

FIG. 5A shows rows of and FIG. 5B shows further rows of an exemplary table for determining a risk score for diabetes.

FIG. 6A shows rows of and FIG. 6B shows further rows of an exemplary table for determining a risk score for chronic obstructive pulmonary disease (COPD).

FIG. 7A shows rows of and FIG. 7B shows further rows of an exemplary table for determining a risk score for hypertension.

FIG. 8A shows rows of and FIG. 8B shows further rows of an exemplary table for determining a risk score for coronary artery disease (CAD).

FIG. 9A shows rows of and FIG. 9B shows further rows of an exemplary table for determining a risk score for heart failure.

FIG. 10A shows rows of and FIG. 10B shows further rows of an exemplary table for determining a risk score for chronic kidney disease (CKD).

FIG. 11A shows rows of and FIG. 11B shows further rows of an exemplary table for determining a risk score for asthma.

FIG. 12 is an example table used for determining a risk score according to an example risk stratification model, according to embodiments of the technology described herein.

FIG. 13 is a exemplary table showing example values for variables used for determining a risk score, according to embodiments of the technology described herein.

FIG. 14A is a flowchart of an illustrative process for generating a graphical user interface (GUI) including a prompt to a healthcare professional to administer a level of care to a patient, according to embodiments of the technology described herein.

FIG. 14B is an example GUI including a prompt to a healthcare professional to administer a level of care to a patient, according to embodiments of the technology described herein.

FIG. 15 is a block diagram of a computer system on which various functions can be implemented.

### DETAILED DESCRIPTION

According to aspects of the disclosure, there are provided systems and method for determining patient prioritization scores for use by healthcare professionals in administering care to patients. Healthcare data of patients may be used to determine one or more of a care gap score representing opportunities to improve care of a patient, a risk score representing risks to health of the patient, a change over time in the risk score for the patient, a measure representing a willingness of the patient to receive care, and a prediction of future costs associated with care of the patient. The one or more scores and measures may be used to determine a patient prioritization score for the patient. A healthcare professional may be prompted based on the patient prioritization score to administer of care to the patient. A graphical user interface (GUI) may be generated that includes the prompt, and the GUI may be provided to a display.

Healthcare insurers and healthcare providers (which may be jointly referred to herein as “Providers”) have a vested interest in targeting patients for health improvement. This interest is greatly enhanced by the shift to value-based care reimbursement strategies. In contrast to traditional fee-for-service models that prioritize the quantity of health services delivered, value-based care reimbursement strategies focus on the quality of care provided. As a result, Providers subject to such value-based care reimbursement strategies are incentivized to invest more time and resources into helping patients to prevent and/or recover from illnesses and injuries more quickly, and to avoid chronic diseases altogether, thereby improving patient outcomes.

The inventors have recognized that, due to limited time and limited resources, it is unrealistic and unnecessary for Providers to provide the same level of care to every patient. First, the quality of services would suffer due to the large volume of patients (e.g., greater than 500,000 patients), hindering the ability of Providers to positively impact patient outcome, thereby negatively impacting both profit and long-term health of patients. Second, there is a wide spectrum of patients ranging from very healthy to very sick. Using resources on those who will not benefit from them (e.g., very healthy patients, terminally ill patients, uncooperative patients, etc.) is wasteful and detrimental, given the shortage of healthcare staff and significant resource constraints. For example, some patients may be at low risk for certain health conditions, less willing to follow medical guidance, and/or less likely to responsive to available care. Healthcare services are less likely to significantly impact the health outcomes of such patients.

Accordingly, the inventors have developed systems and methods that enable Providers to quickly identify patients most likely to be impacted by the available healthcare services, and who should be prioritized in receiving those services. For example, the techniques developed by the inventors may enable Providers to efficiently provide better or best care by prioritizing those patients who need more or most attention and are likely to be responsive to advice on how to better take care of their health.

Conventional techniques for predicting a level of care to be administered to a patient are not equipped with the infrastructure or methods for analyzing large amounts of healthcare data from disparate sources. As a result, they overlook important patient information by focusing on only a limited number of healthcare factors. For example, existing risk adjustment models rely explicitly on claims data, which can be obtained from a single source such as healthcare insurance providers. While claims data can provide insights into past healthcare visits, it does not provide any information about the patient's lifestyle (e.g., habits such as exercising, smoking, and drinking, mental health history, employment, residence, height, weight, age, etc.), which significantly impacts the health of the patient. As another example, existing social determinants of health score prediction models rely solely on socio-economic data, which can be obtained through a limited number of sources such as patient surveys, but which exclude clinical and claims data. By excluding clinical and claims data, such models ignore direct insights into patients' medical history. Accordingly, existing solutions fail to comprehensively account for the wide variety of factors that influence patient health, and therefore do not accurately predict the extent of healthcare that will be required by the patient in the future.

Accordingly, the inventors have developed techniques that address the above-described challenges associated with existing solutions. In some embodiments, the techniques include determining a Patient Prioritization Score (PPS) for each of one or more patients. In some embodiments, the PPS is indicative of the degree to which a patient should be prioritized with respect to receiving healthcare services. As a non-limiting example, a patient with a high PPS may represent a patient with one or more of the following exemplary characteristics: likely to be in a clinical state where their health can be impacted (may not be the highest risk patient), has numerous care gaps (so provides multiple opportunities to impact health), has an increased risk to certain diseases and/or conditions (so is in danger of getting worse), likely to follow health advice, and likely to cost more to provide care to in the future. When administering care, Providers may prioritize administration of care to those patients with a high PPS, as opposed to those patients with a low PPS. Accordingly, in some embodiments, it may be helpful to determine a PPS for each of multiple prospective patients, and then to rank those patients according to the determined PPS s. For example, a PPS may be determined for each of at least 2, at least 5, at least 10, at least 25, at least 50, at least 100, at least 500, at least 1,000, at least at least 10,000, at least 100,000, at least 500,000, at least 1 million, at least 10 million, between 2 and 10 million, between 100 and 100,000, or any other suitable number of patients, as aspects of the technology are not limited in this respect.

In some embodiments, a PPS is determined based on one or more factors. For example, patients may be prioritized based on one or more of the following factors: a score based on gaps in the patient's care (“care gap score”), a risk score, changes in the risk score over time, a measure of the patient's willingness to accept guidance and intervention, and a prediction of future costs associated with the patient's care. In some embodiments, values may be assigned to one or more of the factors, and the values may be used to determine the PPS. For example, the PPS may be determined by determining a linear or non-linear combination of the values, or by providing the values and/or patient healthcare data as input to a machine learning model trained to predict the PPS. By evaluating data associated with multiple healthcare factors, the techniques developed by the inventors are more comprehensive and therefore an improvement to conventional techniques, which focus on only one or a limited number of factors. Because they are more comprehensive, they enable a more accurate prediction of current and future healthcare needs of different patients, which enables Providers to more effectively allocate resources and administer the appropriate care to address these needs.

The techniques developed by the inventors for determining patient prioritization scores improve the field of value-based healthcare because they identify patients who should be prioritized in receiving healthcare resources. As described above, value-based care models focus on improving the quality, as opposed to quantity, of care delivered. In some embodiments, patient prioritization scores determined according to the techniques developed by the inventors can be used to identify those patients who should be prioritized in terms of administering healthcare and allocating healthcare resources. For example, patients with high patient prioritization scores may be prioritized by healthcare providers in providing different types of healthcare services. Such patients may represent those who would benefit most from the administered healthcare. This enables the efficient allocation of resources; resources are not wasted on those patients who do not need them or will not benefit from them, or those patients who are not responsive (e.g., do not respond to healthcare providers, are not compliant with medications, etc.).

In some embodiments, the techniques include obtaining data for each of the multiple healthcare factors and storing the data in an efficient manner. Since each of the factors addresses a different aspect of patient health, the data is obtained from a variety of disparate sources. For example, data from different sources may be processed for determining each of the care gap score, the risk score, the change in risk score, the measure representing patient willingness, and the prediction of future costs. Such sources may include various different types of Providers (e.g., healthcare insurance providers, clinicians, physicians, psychiatrists, dentists, pharmacists, psychologists, therapists, etc.), surveys, patients themselves, and/or any other suitable sources. In some embodiments, once the data has been obtained, it is stored (e.g., in a data store) in a manner that enables efficient processing of such data for determining the different healthcare factors and for ultimately determining the PPS for one or more patients. Obtaining, storing, and processing such massive amounts of healthcare data for each of one or more (e.g., tens to millions) patients from several different sources to determine a PPS is a massive computational burden that cannot be performed in the human mind.

The inventors have recognized that one challenge associated with determining the PPS is the availability of data. For example, a wide variety and large volume of healthcare data is needed to determine values for the one or more factors used to determine the PPS. Some of this data may be obtained by Providers based on medical reports, self-reported healthcare data, and/or by requesting missing data from patients themselves. This is impractical, burdensome, and noncomprehensive approach for various reasons. First, requesting data from patients is burdensome on both patients and Providers, especially when data is needed for hundreds to millions of patients. Different patients will have different data gaps, and thus each request for missing data would need to be patient-specific, making the process of preparing the requests very time-consuming and resource intensive. Second, if a patient has not discussed a particular clinically-relevant issue with their Provider(s), then that issue may be overlooked and excluded from the data, which may impact the value determined for a particular factor (e.g., risk score, care gap score, prediction of future costs, etc.) and the overall PPS. For example, a patient may be experiencing pain in a part of their body. Without discussing the pain with a Provider, it will be excluded from the data, in addition to any healthcare data resulting from follow-up questions by the Provider. As a result, the future costs associated with treating the pain, the increased risk of a more serious injury, and the lack of treatment of the pain will be overlooked in determining the PPS.

Accordingly, the inventors have developed systems and methods for identifying and collect clinically-relevant patient information for filling data gaps that address the above-described challenges. In some embodiments, the systems and methods include generating prompts, using a machine learning model (e.g., a large language model), to identify and collecting clinically-relevant information from patients. In some embodiments, the techniques include providing the machine learning model with existing patient information. The machine learning model may use the patient information to generate patient-specific prompts, which are output to a patient (e.g., via a graphical user interface (GUI), short message service (SMS) messages, instant message, etc.). For example, the prompts may request data for filling data gaps, request follow-up information with respect to the patient's condition(s), ask for input via survey (e.g., to assess patient willingness), or include any other suitable requests or questions, as aspects of the technology are not limited in this respect. In some embodiments, the patient may provide responses to the prompts (e.g., via the GUI, SMS messages, instant messages, etc.). In some embodiments, the patient responses are stored (e.g., in memory, in the Cloud, etc.). In some embodiments, the patient responses are provided as input to the machine learning model, which generates follow-up prompts based on the patient responses.

Accordingly, the techniques developed by the inventors enable a less burdensome and more comprehensive approach for filling data gaps and identifying clinically-relevant information. By utilizing machine learning models that are provided with patient-specific data, the techniques reduce the resource-intensive burden on Providers caused by the need to review data for each patient, identify data gaps, and generate personalized requests for filling those gaps. The techniques also increase the efficiency of gathering data and generating PPSs by reducing, if not eliminating, the need for back-and-forth communication between patients and Providers when filling data gaps. Furthermore, because the machine learning model has access to vast amounts of data, in addition to patient-specific data and patient responses, the machine learning model can identify and explore clinical issues that would have been overlooked in a simple questionnaire, thereby generating a more comprehensive set of healthcare data on which the PPS is based.

Following below are descriptions of various concepts related to, and embodiments of, techniques for determining a patient prioritization score for a patient. It should be appreciated that various aspects described herein may be implemented in any of numerous ways. Examples of specific implementations are provided herein for illustrative purposes only. In addition, the various aspects described in the embodiments below may be used alone or in any combination and are not limited to the combinations explicitly described herein.

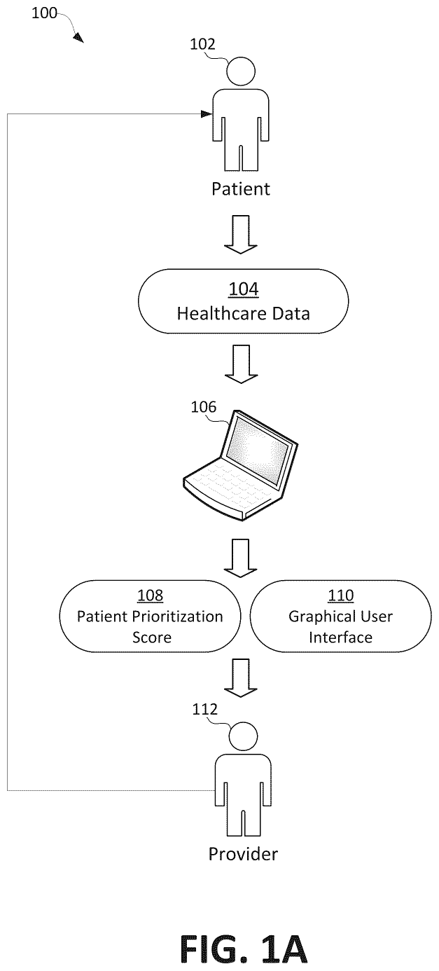

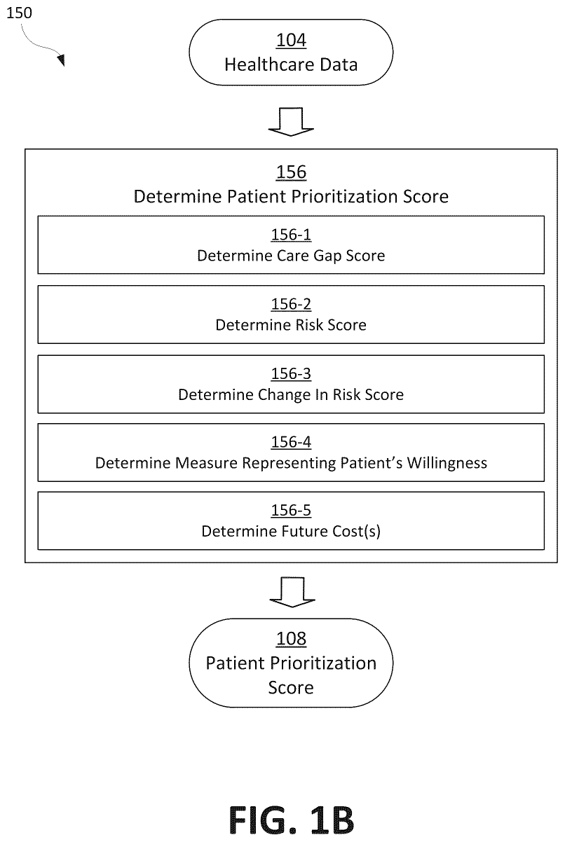

FIG. 1A is a diagram depicting an illustrative technique 100 for determining a patient prioritization score of a patient, according to some embodiments of the technology described herein. As shown, illustrative technique 100 includes processing healthcare data 104 from patient 102 using computing device 106 to determine a patient prioritization score (PPS) 108 for the patient. In some embodiments, the healthcare data 104, PPS 108, and/or any other suitable information (e.g., data derived from the healthcare data 104 and/or PPS 108) may be used to generate a graphical user interface (GUI) 110. In some embodiments, the healthcare data 104 and/or PPS 108 may be output to provider(s) 112 (e.g., via the GUI 110), who may administer care to the patient 102 based on the healthcare data 104 and/or PPS 108.

In some embodiments, aspects of the illustrated technique 100 may be implemented in a clinical setting (e.g., hospital, clinic, nursing home, mental health and addiction treatment center, birth center, hospice care facility, dialysis facility, imaging and radiology center, etc.) or a financial setting (e.g., health insurance company, bank, etc.). For example, aspects of the technique 100 may be implemented on a computing device 106 that is located within a clinical or financial setting. In some embodiments, the computing device 106 may receive healthcare data 104 directly from a user in the clinical or financial setting. For example, patient 102 or provider 112 may enter the healthcare data 104 into the computing device 106 using a user interface of the computing device 106. In some embodiments, the computing device 106 may receive healthcare data 104 indirectly from another device that is located externally from or is co-located with the computing device within the clinical or financial setting. For example, computing device 106 may obtain the healthcare data 104 via at least one communication network, such as the Internet or any other suitable communication network(s) as aspects of the technology described herein are not limited in this respect.

In some embodiments, aspects of the technique 100 may be implemented remote from a clinical or financial setting (e.g., on external server(s)). In this case, the computing device 106 may indirectly obtain healthcare data 104. For example, the healthcare data 104 may be provided to the computing device 106 via at least one communication network, such as the Internet or any other suitable communication network(s), as aspects of the technology are not limited in this respect.

As shown in FIG. 1A, the technique 100 involves obtaining healthcare data 104 from a patient 102. The healthcare data 104 may include any suitable healthcare data, as aspects of the technology are not limited in this respect. As nonlimiting examples, the healthcare data 104 may include demographic data (e.g., age, sex race, ethnicity, nationality, nativity/time in a country, language, etc.), socioeconomic data (e.g., household income, education, employment, food insecurity, housing insecurity, crime prevalence, transportation issues, household composition (e.g., whether a patient lives alone), etc.), attitudes and values (e.g., risk tolerance, attitudes about insurance and doctors, past experiences with healthcare providers, etc.), community data (e.g., urban-rural location of residence, point of care, etc.), access to care (e.g., public and private insurance coverage eligibility, usual source of care, unmet need, etc.), diagnoses, social history, family history, medical visit data, radiology, procedures, lab values, lab trends, lab deltas, medications, follow up, genetic markers, exercise habits, shopping habits, patient survey answers, diet, disabilities and impairments, mental health history, addiction treatment history, medical expense data, and other clinical and lifestyle information. In some embodiments, healthcare data 104 may vary depending on patient 102. For example, if patient 102 has a particular condition, then the healthcare data may include data relating to that condition. However, if another patient does not have that particular condition, then healthcare data obtained for that other patient will not include data relating to the particular condition. Additional examples of healthcare data 104 are described herein.

In some embodiments, the computing device 106 is used to process the healthcare data 104. The computing device 106 may be operated by a user such as provider, a patient, and/or any other suitable entity. For example, the provider may provide the healthcare data 104 as input to the computing device 106 (e.g., by uploading a file) and/or may provide user input specifying processing or other methods to be performed using the healthcare data 104.

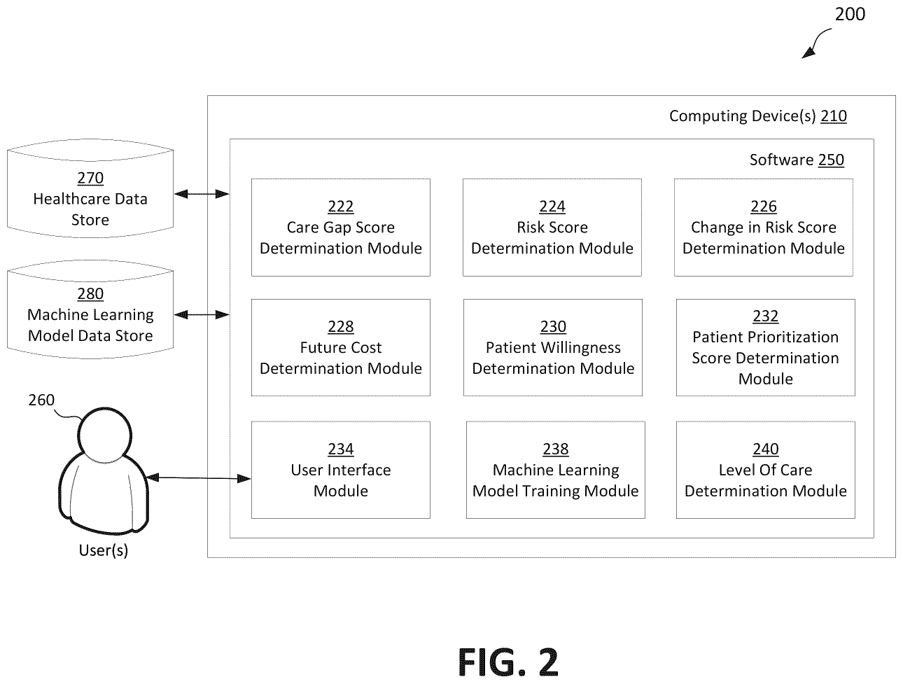

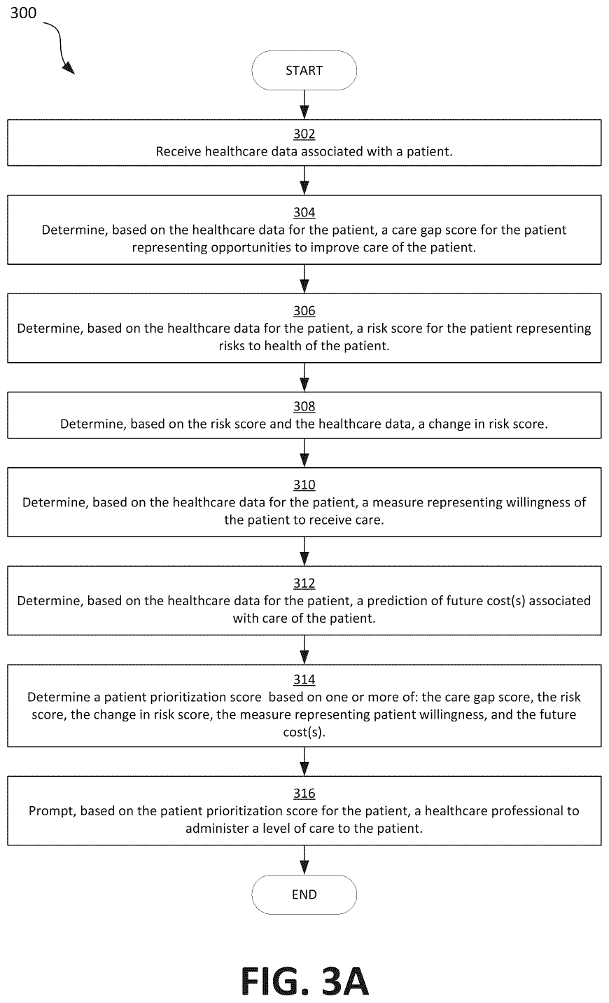

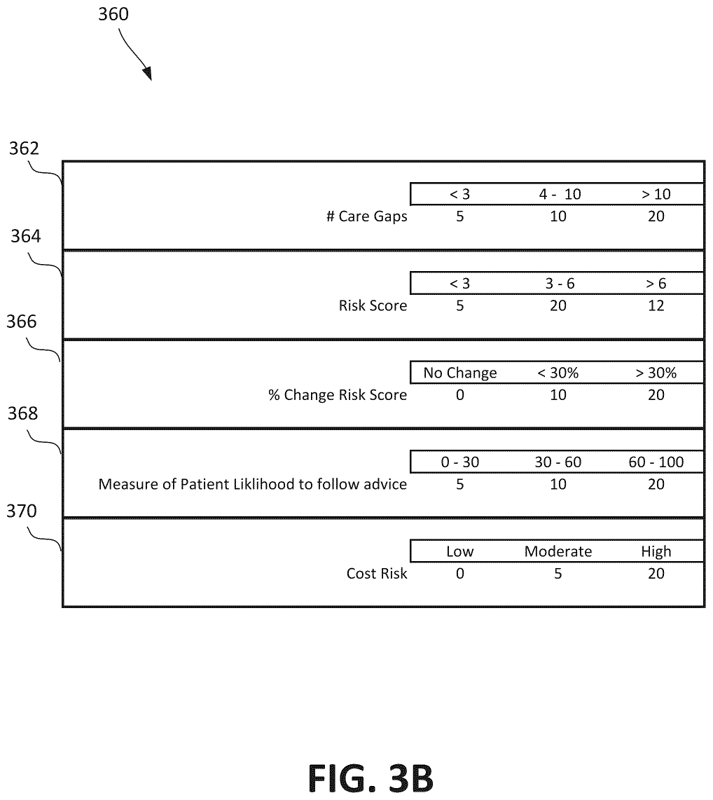

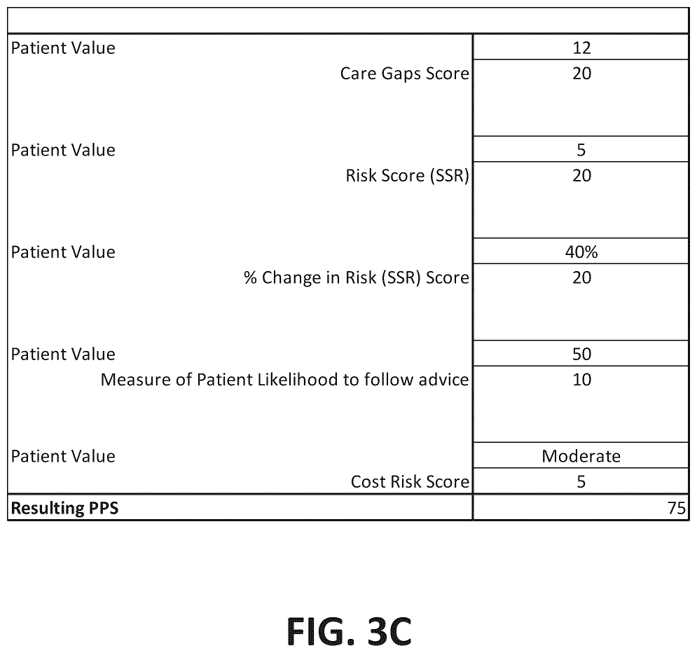

In some embodiments, software on the computing device 106 may be used to determine the PPS 108 for the patient 102. An example of computing device 106 and such software is described herein including at least with respect to FIG. 2 (e.g., computing device 210 and software 250). In some embodiments, software on the computing device 106 may be configured to process at least some of the healthcare data 104 (e.g., all of the healthcare data 104 or less than all of the healthcare data 104) to determine the PPS 108 for patient 102. In some embodiments, this may include: (a) determining, based on the healthcare data 104, a care gap score for the patient 102 representing opportunities to improve care of the patient 102; (b) determining, based on the healthcare data 104, a risk score for the patient 102 representing risks to the health of the patient 102; and (c) determining, based on the care gap score and the risk score, a PPS 108 of the patient 102. Example techniques for determining a PPS are described herein including at least with respect to FIG. 1B and FIG. 3A-3C.

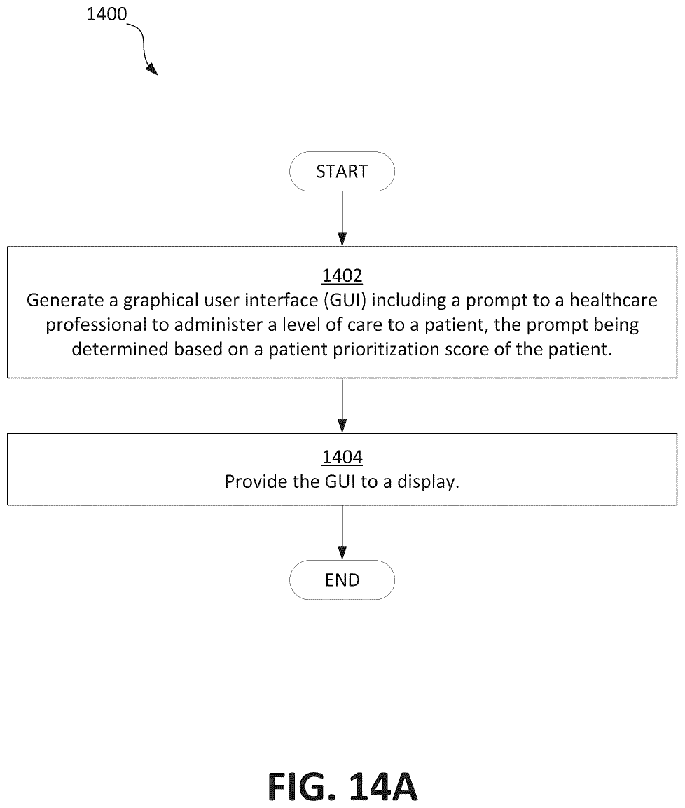

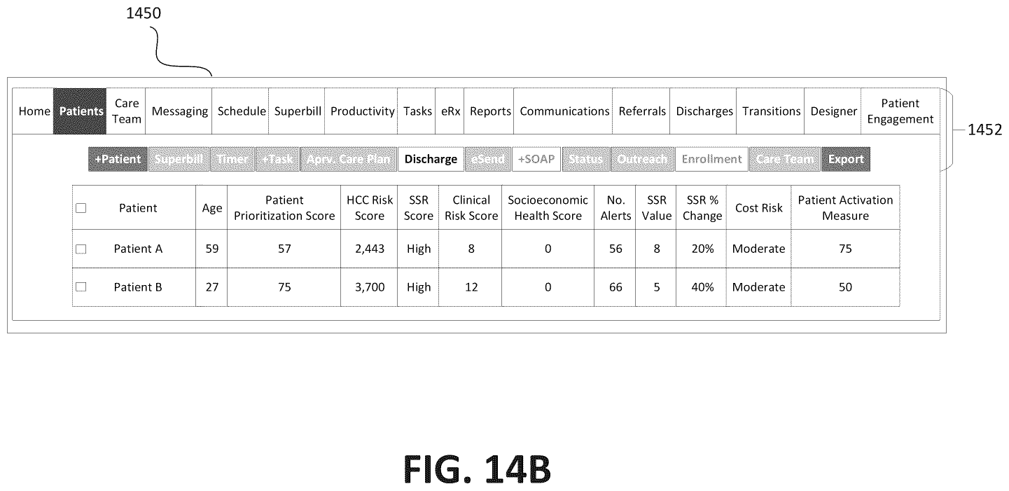

In some embodiments, software on the computing device 106 may prompt a healthcare professional (e.g., provider 112) to administer a level of care to the patient 102. For example, the software may generate GUI 110, which includes a prompt to the healthcare professional (e.g., provider 112) to administer a level of care to the patient. In some embodiments, the prompt is based on the PPS 108 determined for the patient 102. In some embodiments, after generating the GUI, the software provides the GUI to a display, such as a display on computing device 106. Example techniques for generating a GUI are described herein including at least with respect to FIG. 14A.

Additionally, or alternatively, in some embodiments, the PPS 108 is stored (e.g., in memory), transmitted to one or more other devices, or otherwise processed using any suitable techniques, as aspects of the technology described herein are not limited in this respect.

In some embodiments, computing device 106 includes one or multiple computing devices. In some embodiments, when the computing device 106 includes multiple computing devices, each of the computing devices may be used to perform the same process or processes. For example, each of the multiple computing devices may include software used to implement the one or more of the processes 300, 400, and 1400, described herein including at least with respect to FIGS. 3A, 4, and 14A, respectively. In some embodiments, when the computing device 108 includes multiple computing devices, the computing devices may be used to perform different processes or different aspects of a process. For example, one computing device may include software used to process the healthcare data 104 to determine PPS 108, while a different computing device may be used to generate the GUI 110.

In some embodiments, when the computing device 106 includes multiple computing devices, the multiple computing devices may be configured to communicate via at least one communication network, such as the Internet or any other suitable communication network(s), as aspects of the technology described herein are not limited in this respect. For example, one computing device may be configured to receive healthcare data, and then provide the healthcare data to one or more other computing devices via the communication network.

In some embodiments, the technique 100 further includes a step for administering care to the patient 102. For example, provider 112 may administer care to the patient 102. In some embodiments, the level of care administered to the patient is based on the PPS 108. For example, the provider 112 may administer the level of care identified in a prompt displayed via GUI 110.

It should be appreciated that, while illustrative technique 100 shows determining PPS 108 for one patient 102, technique 100 may be repeated for more than one patient, sequentially or in parallel. For example, illustrative technique 100 may be repeated to determine a PPS for each of at least 2, at least 5, at least 10, at least 25, at least 50, at least 100, at least 500, at least 1,000, at least 5,000, at least 10,000, at least 100,000, at least 500,000, at least 1 million, at least million, between 2 and 10 million, between 100 and 100,000, or any other suitable number of patients, as aspects of the technology are not limited in this respect.

FIG. 1B is a diagram depicting an illustrative technique 150 for determining the patient prioritization score (PPS) shown in FIG. 1A, according to some embodiments of the technology described herein. In some embodiments, the illustrative technique 150 includes determining, at act 156, the PPS 108 based on healthcare data 104.

In some embodiments, the technique 150 is implemented using computing device 106. For example, computing device 106 may be configured to receive healthcare data 104 as input and to provide the PPS 108 as output. For example, as described herein including at least with respect to FIG. 1A, the computing device 106 may be configured to generate a GUI that conveys the PPS 108 to a provider.

In some embodiments, determining the PPS at act 156 includes: (a) determining a care gap score (act 156-1); (b) determining a risk score (act 156-2); (c) determining a change in risk score (act 156-3); (d) determining a measure representing the patient's willingness (act 156-4); (e) determining a prediction of future cost(s) (act 156-5); and (f) determining the PPS based on one or more of the determined risk score, change in risk score, measure representing the patient's willingness, and/or prediction of future costs. Example techniques for determining each of the foregoing are described herein including at least with respect to FIG. 3A and the sections “Care Gap Score,” “Risk Score,” “Change in Risk Score,” “Patient Willingness,” and “Prediction of Future Cost(s)”. It should be appreciated that, in some embodiments, one or more of acts 156-1, 156-2, 156-3, 156-4, and 156-5 may be optional. For example, a subset of the acts may be performed, and the results used to determine the PPS. As one example, determining the PPS at act 156 may include: (a) determining the care gap score; (b) determining the risk score; and (c) determining the PPS based on the determined care gap and risk scores. Additionally, it should be appreciated that acts 156-1, 156-2, 156-3, 156-4, and 156-5 may be performed in any order and/or in parallel.

In some embodiments, determining the PPS includes combining the determined care gap score, risk score, change in risk score, measure representing the patient's willingness, and/or prediction of future costs to obtain a single value representing the PPS for the patient. For example, in some embodiments, the PPS may be determined by determining a linear or non-linear combination of the numerical values representing one or more of the foregoing factors.

In an alternative embodiment, the healthcare data 104 may be provided to a machine learning model trained to determine the PPS 108. For example, the data used to train the machine learning model may include healthcare data that would be used to determine the care gap score, risk score, change in risk score, measure representing the patient's willingness, and/or prediction of future cost(s), in connection with the PPS s determined according to act 156 based on the provided healthcare data. Using a machine learning model may improve efficiency of determining the PPS when processing large volumes of healthcare data. Further techniques for determining a PPS are described herein including at least with respect to FIG. 3A.

FIG. 2 is a block diagram of an example system 200 for determining a patient prioritization score for a patient, according to some embodiments of the technology described herein. System 200 includes computing device 210 that is configured to have software 250 execute thereon to perform various functions in connection with determining a PPS for a subject and/or generating a GUI that includes a prompt to a healthcare provider to administer a level of care to the subject.

The computing device 210 can be one of multiple computing devices of any suitable type. For example, the computing device 210 may be a portable computing device (e.g., laptop, a smartphone) or a fixed computing device (e.g., a desktop computer, a server). When computing device 210 includes multiple computing devices, the device(s) may be physically co-located (e.g., in a single room) or distributed across multiple physical locations. In some embodiments, the computing device 210 may be part of a cloud computing infrastructure.

In some embodiments, the computing device 210 may be operated by one or more user(s) 260 such as one or more Provider(s), patient(s), and/or other individual(s). For example, the user(s) 260 may provide healthcare data as input to computing device 210 (e.g., by uploading one or more files), and/or may provide user input specifying processing or other methods to performed on the healthcare data.

As shown in FIG. 2, software 250 includes a plurality of modules. Each module may include processor-executable instructions that, when executed by at least one computer hardware processor, cause the at least one computer hardware processor to perform the function(s) of that module. Such modules are sometimes referred to herein as “software modules.” The software modules shown in FIG. 2, include processor-executable instructions that, when executed by a computing device, cause the computing device to perform one or more processes, such as the processes described herein including at least with respect to FIGS. 3A, 4, and 14A. It should be appreciated that the modules shown in FIG. 2 are illustrative and that, in other embodiments, software 250 may be implemented using one or more other software modules, in addition to or instead of, the modules shown in FIG. 2. In other words, the software 250 may be organized internally differently from how illustrated in FIG. 2.

As shown in FIG. 2, software 250 includes multiple software modules for processing at least some healthcare data, such as care gap score determination module 222, risk score determination module 224, change in risk score determination module 226, future cost determination module 228, patient willingness determination module 230, patient prioritization score determination module 232, and level of care determination module 240. In the embodiment of FIG. 2, the software 250 additionally includes a user interface module 234 and machine learning model training module 238.

In some embodiments, each of the care gap score determination module 222, risk score determination module 224, change in risk score determination module 226, future cost determination module 228, and patient willingness determination module 230 obtains healthcare data from healthcare data store 270 and/or user(s) 260 (e.g., by the user uploading the healthcare data). The healthcare data store 270 may be of any suitable type (e.g., database system, multi-file, flat file, etc.) and may store healthcare data in any suitable way and in any suitable format, as aspects of the technology described herein are not limited in this respect. The healthcare data store 270 may be part of or external to computing device(s) 210.

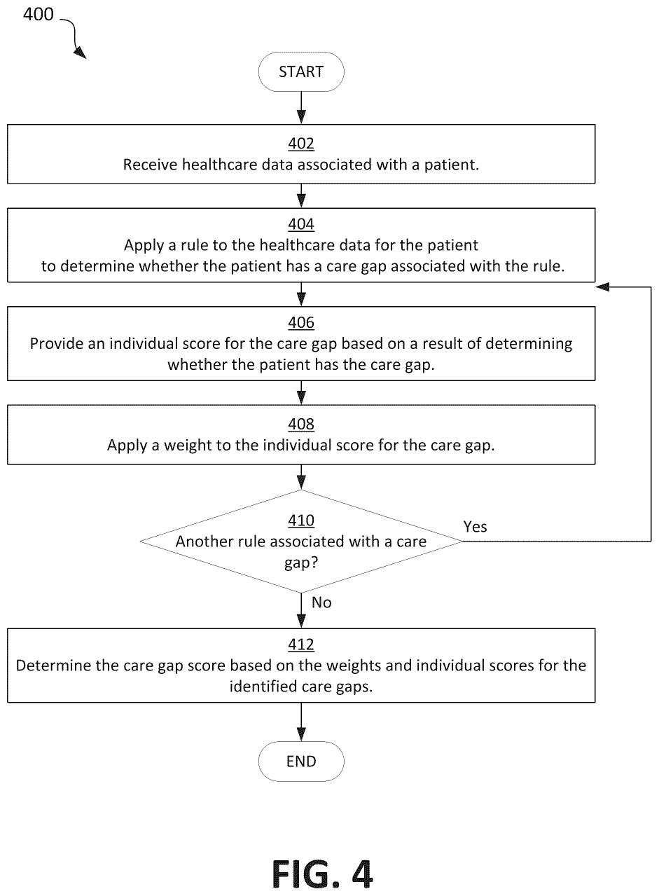

In some embodiments, the care gap score determination module 222 is configured to determine a care gap score for the patient. For example, the care gap score determination module 222 may be configured to process at least some of the healthcare data to determine the care gap score. As nonlimiting examples, the care gap score determination module 222 may be configured to process data including demographics, diagnoses, social history, family history, medical visit data, radiology, procedures, lab values, lab trends, lab deltas, medication, follow up, genetic markers, exercise habits, shopping habits, household income, education, employment, food insecurity, housing insecurity, crime prevalence, transportation issues, household composition (e.g., whether a patient lives alone), and other clinical and lifestyle information. In some embodiments, the care gap score determination 222 may be configured to apply a rule-based algorithm to the healthcare data to determine the care gap score. In some embodiments, the care gap score determination module may process the healthcare data using a machine learning model trained to predict the care gap score. For example, the machine learning model may be obtained from the machine learning model data store 280. Example techniques for determining a care gap score are described herein including at least with respect to FIG. 3A, FIG. 4, and the section “Care Gap Score”.

In some embodiments, the care gap score determination module 222 is further configured to identify and/or obtain clinically-relevant healthcare data from the patient. For example, the care gap score determination module 222 may be configured to use a machine learning model, such as a large language model, to generate prompts requesting data from a user (e.g., user(s) 260). In some embodiments, the requested data includes missing data needed for determining the care gap score. In some embodiments, the prompts are responsive to user input and/or existing healthcare data. For example, the machine learning model may process the user input and/or existing healthcare data and generate prompts that include follow-up questions for obtaining additional information from the user, thereby generating a more comprehensive set of data for determining the care gap score. In some embodiments, the care gap score determination module 222 be configured to obtain such a machine learning model from the machine learning model data store 280.

In some embodiments, the risk score determination module 224 is configured to determine a risk score for the subject. For example, the risk score determination module 224 may be configured to process at least some of the healthcare data to determine the risk score. As nonlimiting examples, the risk score determination module 224 may be configured to process data including medical visit data, lab values, lab trends, lab deltas, medications, vaccinations, diagnoses, lifestyle habits (e.g., smoking), medical history, demographics, mental health conditions, medical device use, treatments, social history, family history, genetic markers, exercise habits, household income, education, employment, food insecurity, housing insecurity, crime prevalence, transportation issues, household composition (e.g., whether a patient lives alone), and/or any other suitable healthcare data.

In some embodiments, the risk score determination module 224 is configured to process the healthcare data using any suitable risk score model. Non-limiting examples of risk score models include Persivia's Soliton Suggested Risk Score (SSR) model, the Social Determinants of Health (SDOH) risk score, and the American Academy of Family Physicians (AAFP) risk stratification score. However, any other suitable risk score model may be used to predict a risk score for the patient, as aspects of the technology described herein are not limited in this respect. Example techniques for determining a risk score are described herein including at least with respect to FIG. 3A and the section “Risk Score”.

In some embodiments, the change in risk score determination module 226 is configured to process risk score data from risk score determination module 224 to determine a change in risk score for the patient. In some embodiments, the risk score data may include multiple risk scores (e.g., 2 or more risk scores) determined at different time points. As one example, the risk score data may include a current risk score and one or more risk scores determined at earlier time point(s) (e.g., a time point days, weeks, months, or years earlier). In some embodiments, the change in risk score is represented by any suitable measure of change, as aspects of the technology is not limited in this respect. For example, the change in risk score may reflect a percent change in risk score, a difference between two risk scores, a slope of a line fitted to multiple risk scores over time, or any other suitable measure. Example techniques for determining a change in risk score are described herein including at least with respect to FIG. 3A and the section “Change in Risk Score”.

In some embodiments, the future cost determination module 228 is configured to predict cost(s) associated with future care of the patients (e.g., future cost(s)). For example, the future cost determination module 228 may be configured to process at least some of the healthcare data to determine the prediction of future cost(s) associated with care of the patient. As nonlimiting examples, the future cost determination module 228 may be configured to process claims data (e.g., data used to process insurance claims) such as medical visit data, diagnoses, treatments, procedures, medications, and/or any other suitable healthcare data.

In some embodiments, the future cost determination module 228 is configured to process the healthcare data using any suitable cost risk prediction model. Non-limiting examples of cost risk prediction models include Centers for Medicare and Medicaid Services Hierarchical Condition Category (CMS-HCC) risk adjustment model and the Johns Hopkins ACG System. However, any other suitable cost risk prediction model may be used to predict future cost(s) for the patient, as aspects of the technology described herein are not limited in this respect. Example techniques for predicting future cost(s) are described herein including at least with respect to FIG. 3A and the section “Prediction of Future Cost(s).

In some embodiments, the patient willingness determination module 230 is configured to determine a measure representing patient willingness. In some embodiments, the measure may represent a patient's willingness to follow instructions and/or to perform activities that are beneficial to their health. As non-limiting examples, the measure may reflect patient willingness to comply with a medication dosage schedule, responsiveness to communications from providers, willingness to receive routine medical examinations, willingness to undergo certain procedures, reception to preventative care (e.g., vaccines), and willingness to visit specialists.

In some embodiments, the patient willingness determination module 230 is configured to process at least some of the healthcare data to determine the measure representing patient willingness. As nonlimiting examples, the healthcare data may include the patient's answers to a survey regarding the patient's attitude toward taking charge of their own treatment. For example, the answers may indicate whether the patient agrees or disagrees with statements included in the survey. In some embodiments, each statement has a numerical score associated with each of the various possible responses, and the numerical values are used to determine the final measure representing patient willingness. For example, the patient willingness determination module 230 may determine a weighted or unweighted sum of the scores based on the patient's responses. Example techniques for determining a measure representing patient willingness are described herein including at least with respect to FIG. 3A and the section “Patient Willingness”.

In some embodiments, the patient willingness determination module 230 is further configured to obtain responses from the patient relating to patient willingness. For example, the patient willingness determination module 230 may be configured to use a machine learning model, such as a large language model, to generate prompts requesting input from a user (e.g., user(s) 260). In some embodiments, the requested input includes responses to survey questions relating to the patient's attitude about taking charge of their own treatment. In some embodiments, the prompts are responsive to user input and/or existing healthcare data. For example, the machine learning model may process the user input and/or existing healthcare data and generate prompts that include follow-up questions for obtaining additional information from the user, thereby generating a more comprehensive set of data for determining the measure representing patient willingness. In some embodiments, the patient willingness determination module 230 be configured to obtain such a machine learning model from the machine learning model data store 280.

In some embodiments, the patient prioritization score (PPS) determination module 232 is configured to determine a PPS for a patient, such as user(s) 260. In some embodiments, the PPS determination module 232 is configured to determine the PPS using one or more of: a care gap score obtained from the care gap score determination module 222, a risk score obtained from the risk score determination module 224, a change in risk score obtained from the change in risk score determination module 226, a prediction of future cost(s) obtained from the future cost determination module 228, and a measure of patient willingness obtained from the patient willingness determination module 230. In some embodiments, the PPS determination module 232 is configured to assign a value to each of the above-listed factors. As one non-limiting example, the PPS determination module 232 may be configured to assign a value between 0 and to the above-listed factors. In some embodiments, the PPS determination module 232 is configured to determine the PPS based on the value assigned to the factor(s). For example, the PPS determination module may be configured to determine a linear or non-linear sum of the values.

In some embodiments, the PPS determination module 232 is configured to determine the PPS by processing at least some of the healthcare data (e.g., obtained from healthcare data store 270, via user interface 234, etc.) using a machine learning model. For example, the machine learning model may include a machine learning model obtained from machine learning model data store 280. In some embodiments, the machine learning model is configured to process the healthcare data processed by one or more of: the care gap score determination module 222, risk score determination module 224, change in risk score determination module 226, future cost determination module 228, and patient willingness determination module 230. In some embodiments, the machine learning model is trained to predict the PPS based on said healthcare data. Example techniques for determining a PPS are described herein including at least with respect to FIG. 3A.

In some embodiments, the level of care determination module 240 is configured to determine, based on a PPS obtained from PPS determination module 232, a level of care to be administered to the patient for whom the PPS was determined. For example, in some embodiments, when the PPS is relatively high (e.g., as compared to other PPSs determined for other patients) the level of care determination module 240 may determine that a higher level of care should be administered to that patient (e.g., relative to other patients with lower PPSs). For example, the level of care determination module 240 may organize a list of patients according to the order in which the patients should be prioritized, based on PPSs determined for those patients.

As shown in FIG. 2, in some embodiments, software 250 further includes a user interface module 234. User interface module 234 may be configured to generate a graphical user interface (GUI), a text-based user interface, and/or any other suitable type of interface through which a user may provide input and view information generated by software 250. For example, in some embodiments, the user interface module 234 may be a webpage or web application accessible through an Internet browser. In some embodiments, the user interface module 234 may generate a graphical user interface (GUI) of an app executing on the user's mobile device. In some embodiments, the user interface module 234 may generate a number of selectable elements through which a user may interact. For example, the user interface module 234 may generate dropdown lists, checkboxes, text fields, or any other suitable element.

In some embodiments, the user interface module 234 is configured to generate a GUI. In some embodiments, the user interface module 234 generates the GUI based on: the PPS obtained from the PPS determination module 232, a care gap score obtained from the care gap score determination module 222, a risk score obtained from the risk score determination module 224, a change in risk score obtained from the change in risk score determination module 226, a prediction of future cost(s) obtained from the future cost determination module 228, a measure of patient willingness obtained from the patient willingness determination module 230, healthcare data obtained from the healthcare data store 270 and/or user(s) 260, and/or any other suitable information, as aspects of the technology described herein are not limited in this respect.

In some embodiments, the GUI includes a prompt to a healthcare provider (e.g., user(s) 260) to provide a level of care to a patient. In some embodiments, the user interface module 234 may obtain the level of care from the level of care determination module 240 and include an indication of the level of care in the GUI. For example, the user interface module 234 may generate a GUI that includes a list of patients in the order in which they should be prioritized based on PPS. As another example, the GUI may indicate the level of care in terms of a scale (e.g., a scale of low to high, a numeric scale).

In some embodiments, the user interface module 234 may further identify actions to be taken or recommended by the healthcare provider(s) (e.g., user(s) 260) based on the PPS and level of care. For example, the generated GUI may identify treatments, procedures, medications, change of lifestyle, change in frequency of medical visits, and/or any other suitable action to be taken or recommended by the healthcare provider(s).

In some embodiments, the user interface module 234 may further generate a GUI that displays healthcare data, care gap scores, risk scores, change in risk scores, prediction of future cost(s), measures of patient willingness, and/or PPS s. For example, the user(s) 260 may interact with the GUI (e.g., through user interface module 234) to navigate, modify, and/or select data to be displayed and the manner in which that data is displayed. Techniques for generating a GUI are described herein including at least with respect to FIG. 14A.

In some embodiments, the machine learning model training module 238 may be configured to train one or more machine learning models to perform various tasks. For example, the machine learning model training module 238 may be configured to train a machine learning model to predict a PPS based on healthcare data (e.g., a machine learning model used by PPS determination module 232). Additionally, or alternatively, the machine learning model training module 238 may be configured to train a machine learning model (e.g., a large language model) to generate prompts for acquiring healthcare data for use by the care gap score determination module 222. Additionally, or alternatively, the machine learning model training module 238 may be configured to train a machine learning model (e.g., a large language model) to obtain input from a patient relating to the patient willingness. In these examples, the machine learning model training module 238 may obtain training data from the healthcare data store 270 and/or user(s) 260. Additionally, or alternatively, the machine learning model training module 238 may be configured to train a machine learning model to determine a care gap score based on healthcare data.

In some embodiments, the machine learning model training module 238 may provide machine learning model(s) to the machine learning model data store 280 for storage therein. The machine learning model data store 280 may be of any suitable type (e.g., database system, multi-file, flat file, etc.) and may store machine learning model data in any suitable way and in any suitable format, as aspects of the technology described herein are not limited in this respect. The machine learning model data store 280 may be part of or external to computing device(s) 210.

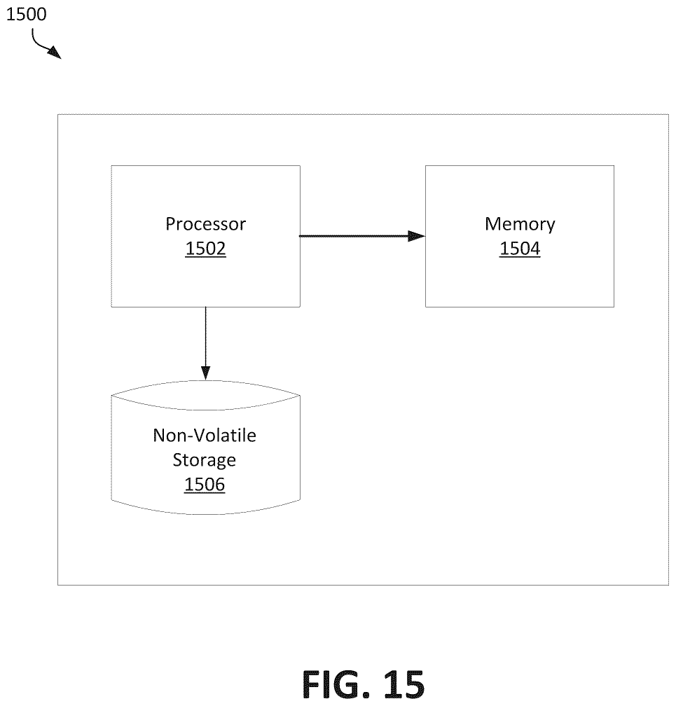

FIG. 3A is a flowchart of an illustrative process 300 for determining a patient prioritization score (PPS) for a patient, according to some embodiments of the technology described herein. Process 300 may be performed by software (e.g., software 250 in FIG. 2) executing on a laptop computer, a desktop computer, one or more servers, in a cloud computing environment, computing device 106 described with respect to FIG. 1A, computing device 210 described with respect to FIG. 2, computing device 1500 described with respect to FIG. 15, or any other suitable computing device.

At act 302, healthcare data associated with the patient is received. The healthcare data may include any suitable healthcare data, as aspects of the technology are not limited in this respect. In some embodiments, healthcare data includes healthcare data 104 described herein with respect to FIGS. 1A and 1B.

In some embodiments, receiving the healthcare data at act 302 includes receiving the healthcare data from one or more users. In some embodiments, the healthcare data is received from one or more Provider(s) and/or patient(s). For example, a Provider and/or patient may enter the healthcare data using a user interface of a computing device (e.g., via user interface module 234 shown in FIG. 2). Additionally, or alternatively, a Provider and/or patient may upload a file that includes the healthcare data. Additionally, or alternatively, a Provider and/or patient may interact with prompts generated by a machine learning model (e.g., a large language model) to provide the healthcare data.

Additionally, or alternatively, in some embodiments, receiving the healthcare data at act 302 includes receiving the healthcare data from a healthcare data store (e.g., healthcare data store 270). For example, the healthcare data may have previously been uploaded, entered, or otherwise provided, and stored in the healthcare data store.

At act 304, a care gap score is determined for the patient based on at least some of the healthcare data received at act 302. In some embodiments, the care gap score represents opportunities to improve care of the patient. Example of care gaps and techniques for determining a care gap score are described herein including at least with respect to the section “Care Gap Score.”

At act 306, a risk score is determined for the patient based on at least some of the healthcare data received at act 302. In some embodiments, the risk score represents risks to health of the patient. Techniques for determining a risk score are described herein including at least with respect to the section “Risk Score.”

At act 308, a change in risk score is determined for the patient based on the risk score determined at act 306 and at least some of the healthcare data received at act 302. For example, the healthcare data received at act 302 may include data relating to one or more risk scores for the patient determined at previous points in time (e.g., days, weeks, months, or years ago). Techniques for determining a change in risk score are described herein including at least with respect to the section “Change in Risk Score.”

At act 310, a measure representing patient willingness is determined for the patient based on at least some of the healthcare data received at act 302. For example, the measure may represent willingness of the patient to receive care. Techniques for determining a measure of patient willingness are described herein including with respect to the section “Patient Willingness.”

At act 312, a prediction of future cost(s) is determined based on at least some of the healthcare data received at act 302. For example, the prediction may represent cost(s) associated with future care of the patient. Techniques for determining a prediction of future cost(s) are described herein including with respect to the section “Prediction of Future Cost(s).”

At act 314, the PPS is determined for the patient. In some embodiments, the PPS is determined based on one or more of: the care gap score determined at act 304, the risk score determined at act 306, the change in risk score determined at act 308, the measure of patient willingness determined at act 310, and the prediction of future cost(s) determined at act 312. For example, the PPS may be determined based on only one of the factors, two of the factors, three of the factors, four of the factors, or all five of the factors.

In some embodiments, determining the PPS includes assigning a value to each of the factors (e.g., the care gap score, risk score, change in risk score, measure of patient willingness, and prediction of future costs). For example, any suitable range(s) of values may be assigned to the factors. An example of assigning values to factors used to determine the PPS is described herein with respect to FIGS. 3B and 3C.

In some embodiments, the values assigned to the factors are used to determine the PPS. For example, in some embodiments, determining the PPS includes determining a weighted or unweighted, linear or non-linear combination of the factors.

In some embodiments, the PPS may be used to inform Providers which patients should be prioritized. For example, in some embodiments, Providers may focus on patients with a high or highest PPS relative to other patients. As one nonlimiting example, a high PPS may represent a patient characterized by combination of the following: likely to be in a clinical state where their health can be impacted (may not be the highest risk patient), has numerous care gaps (so provides multiple opportunities to impact health), has an increased risk to certain diseases and/or conditions (so is in danger of getting worse), likely to follow health advice, and likely to cost more to provide care to in the future.

At act 316, process 300 includes prompting a healthcare professional to administer a level of care to the patient. In some embodiments, the level of care is a level with respect to a scale indicating different levels of care. For example, the scale may indicate that the level of care to be administered is high, medium, or low. Additionally, or alternatively, the scale may be a numerical scale. A higher number with respect to the numerical scale may indicate that a higher level of care is to be administered. In some embodiments, the level of care is represented by the placement of a patient in a list of patients. For example, the patients may be organized according to PPS. Patients with a higher PPS may be administered a higher level of care than patients with a lower PPS.

In some embodiments, prompting the healthcare professional includes generating a graphical user interface (GUI) that includes such a prompt. Techniques for generating a GUI that includes a prompt to the healthcare professional to administer a level of care are described herein including at least with respect to FIG. 14A.

In some embodiments, implementing process 300 may include additional or alternative acts that are not shown in FIG. 3A, as aspects of the technology described herein are not limited to those acts shown in FIG. 3A. For example, process 300 may include a subset of the acts shown in FIG. 3A. For example, process 300 may include all of the acts 302-316, or only some of the acts including acts 302 and 314-316; 302-304 and 314-316; 302-314; 304-316; 306-316; 308-316; 312-316, or any other suitable combination of the acts shown in FIG. 3A.

In some embodiments, though shown sequentially, at least some of acts 302-312 may be implemented in parallel including, for example: acts 302-304; 302-306; 302-306 and 310, 302-306 and 310-312, or any other suitable combination of acts. In some embodiments, though shown sequentially, at least some of acts 302-312 may be performed in any order. For example, any of acts 302-306 and 310-312 may be performed in any order.

In some embodiments, at least some of acts 302-316 of process 300 may be repeated to determine a PPS for each of any suitable number of patients. For example, at least some of acts 302-316 may be performed for each of at least 2, at least 5, at least 10, at least 25, at least 50, at least 100, at least 500, at least 1,000, at least 5,000, at least 10,000, at least 100,000, at least 500,000, at least 1 million, at least 10 million, between 2 and 10 million, between 100 and 100,000, or any other suitable number of patients, as aspects of the technology are not limited in this respect.

FIG. 3B shows an example table for determining a patient prioritization score based on one or more factors, according to some embodiments of the technology described herein. It should be appreciated that the purpose of this example is to aid understanding and is not intended to be limiting.

As described herein, in some embodiments, a PPS is determined based on one or more factors including: a care gap score, a risk score, a change in risk score, a measure representing patient willingness, and prediction of future cost(s). In some embodiments, after determining or otherwise obtaining each of the one or more factors, a value may be assigned to the factor. For example, as shown in FIG. 3B, table 360 includes five rows: (1) row 362 corresponds to assigning a value to a care gap score, (2) row 364 corresponds to assigning a value a risk score, (3) row 366 corresponds to assigning a value associated to a change in risk score, (4) row 368 corresponds to assigning a value associated to a measure representing patient willingness, and (5) row 370 corresponds to assigning a value to a prediction of future costs.

As shown in this example, determining each of the foregoing factors includes assigning, to the factor, a value between 0 and 20. It should be appreciated that the range of 0 to 20 is an example range, and that the factors may be assigned values within any suitable range, as aspects of the technology are not limited in this respect. As nonlimiting examples, the factors may be assigned values between 0 to 5, 0 to 10, 0 to 25, 0 to 50, 0 to 100, 0-200, 0-1,000, or any other suitable range.

Row 362 shows an example for assigning a value to a care gap score. In this example, the care gap score is the number of care gaps identified for the patient. As shown, when the number of care gaps is less 3, a value of 5 is assigned to the care gap score. When the number of care gaps is between 4 and 10, a value of 10 is assigned to the care gap score. When the number of care gaps is greater than 10, a value of 20 is assigned to the care gap score. Example techniques for identifying care gaps are described herein including at least with respect to FIG. 3A and the section “Care Gap Score”.

Row 364 shows an example for assigning a value to a risk score. As shown, when the risk score is less than 3, a value of 5 is assigned to it. When the risk score is between 3 and 6, a value of 20 is assigned to it. When the risk score is greater than 6, a value of 12 is assigned to it. Example techniques for determining a risk score are described herein including at least with respect to FIG. 3A and the section “Risk Score”.

Row 366 shows an example for assigning a value to a change in risk score. In this example, the change in risk score is based on the percentage change in risk score. As shown, when there is no change in risk score, a value of 0 is assigned to the change in risk score. When there is less than a 30% change in risk score, a value of 10 is assigned to the change in risk score. When there is greater than 30% change in risk score, a value of 20 is assigned to the change in risk score. Example techniques for determining the change in risk score are described herein including at least with respect to FIG. 3A and the section “Change in Risk Score”.

Row 368 shows an example for assigning a value to a measure of patient willingness. In this example, the measure represents patient willingness to follow advice (e.g., from a healthcare provider). As shown, when the measure is between 0 and 30, a value of 5 is assigned to it. When the measure is between 30 and 60, a value of 10 is assigned to it. When the measure is between 60 and 100, a value of 20 is assigned to it. Example techniques for determining a measure representing patient willingness are described herein including at least with respect to FIG. 3A and the section “Patient Willingness”.

Row 370 shows an example for assigning a value to the prediction of future cost(s). In this example, the prediction of future cost(s) is based on a measure of cost risk. As shown, when the cost risk is low, a value of 0 is assigned to the prediction of future cost(s). When the cost risk is moderate, a value of 5 is assigned to future cost(s). When the cost risk is high, a value of 20 is assigned to the prediction of future cost(s).

In some embodiments, after the values are assigned to each of the factors, they may be combined to determine the PPS. For example, the PPS may be obtained by determining a weighted or unweighted, linear or nonlinear combination of the assigned values.

FIG. 3C shows an example of determining a PPS for a patient using the example table shown in FIG. 3B.

As shown, the resulting PPS was determined to be 75 and is based on the care gap score, risk score, change in risk score, measure representing patient willingness, and cost risk. The care gap score is obtained by comparing the patient value of 12 to the criteria shown in row 362 of table 360 in FIG. 3A. Since 12 exceeds 10, the care gap score is determined to be 20. The risk score is obtained by comparing the patient value of 5 to the criteria shown in row 364 of table 360 in FIG. 3A. Since 5 falls between 3 and 6, the risk score is determined to be 20. The change in risk score is obtained by comparing the patient value of 40% to the criteria shown in row 366 of table 360 in FIG. 3A. Since 40% is greater than 30%, the change in risk score is determined to be 20. The measure representing patient willingness is obtained by comparing the patient value of 50 to the criteria shown in row 368 of table 360 in FIG. 3A. Since 50 falls between 60 and 100, the measure of patient willingness is determined to be 20. The cost risk is obtained by comparing the patient value of moderate to the criteria shown in row 370 of table 360 in FIG. 3A. Accordingly, the cost risk is determined to be 5.

In the example of FIG. 3B, the PPS is determined by summing the values determined for each factor. In this example, the sum of 20, 20, 20, 10, and 5 is equal to a PPS of 75.

Care Gap Score

Aspects of the technology described herein relate to determining a care gap score for a patient. A care gap may be referred to interchangeably as an Evidence Based Care Gap (EBCG).

In some embodiments, a care gap represents an opportunity for providing care to a patient that will help to improve a patient's health. For example, a care gap may represent a particular action that a patient is not currently taking, but which has been demonstrated to improve the health of similarly-situated patients. Consider, for example, a diabetic patient who does not maintain a blood A1C of under 7%. Diabetics that maintain a blood A1C of under 7% have been shown to avoid certain adverse outcomes such as kidney failure or damage to the eyes and other adverse outcomes. In this example, the diabetic patient has a care gap since there is an opportunity for the patient to avoid adverse outcomes by maintaining a blood A1C under 7%. Consider, as another example, a patient who does not exercise and who eats foods high in saturated fats. People who exercise and eat foods high in saturated fats and refined sugars have higher chances of heart disease and diabetes. In this example, the patient has a care gap since there is an opportunity to lower the patient's chance of heart disease and diabetes by exercising and avoiding foods high in saturated fats and refined sugars.

Additional examples of activities demonstrated to improve a person's health include, but are not limited to: diabetics that carefully and regularly monitor their blood sugar; patients with a chronic condition taking chronic condition medications regularly; patients with adult major depressive disorder (MDD) taking a Suicide Risk Assessment; obtaining preventative care and screening (e.g., influenza immunization, pneumococcal vaccination for older adults, breast cancer screening, colorectal screening, etc.); avoiding antibiotic treatment for acute bronchitis/bronchiolitis; diabetic patients obtaining an eye exam; patients with coronary artery disease (CAD) taking Angiotensin-Converting Enzyme (ACE) Inhibitor or Angiotensin Receptor Blocker (ARB) therapy; patients with diabetes mellitus obtaining: diabetic foot and ankle care and evaluations of footwear for ulcer prevention; obtaining body mass index BMI) screening and follow-up plans; diabetic patients obtaining: glucose screening & control, lipid screening & control, blood pressure screening & control, and nephropathy screening & control, eye and foot care.

In some embodiments, the techniques described herein include determining a care gap score for a patient. In some embodiments, the care gap score reflects a number and/or type(s) of care gap(s) identified for a patient. For example, a higher care gap score may represent a greater number of care gap(s) and/or care gap(s) that, if addressed, could significantly improve the patient's health.

FIG. 4 is a flowchart of an illustrative process for determining a care gap score for a patient, according to some embodiments of the technology described herein. Process 400 may be performed by software (e.g., software 250 in FIG. 2) executing on a laptop computer, a desktop computer, one or more servers, in a cloud computing environment, computing device 106 described with respect to FIG. 1A, computing device 210 described with respect to FIG. 2, computing device 1500 described with respect to FIG. 15, or any other suitable computing device.

At act 402, healthcare data associated with the patient is received. The healthcare data may include any suitable healthcare data, as aspects of the technology described herein are not limited in this respect. Nonlimiting examples of healthcare data that may be received at act 402 include demographic data (e.g., age, sex race, ethnicity, nationality, nativity/time in a country, language, etc.), socioeconomic data (e.g., household income, education, employment, food insecurity, housing insecurity, crime prevalence, transportation issues, household composition (e.g., whether a patient lives alone), etc.), attitudes and values (e.g., risk tolerance, attitudes about insurance and doctors, past experiences with healthcare providers, etc.), community data (e.g., urban-rural location of residence, point of care, etc.), access to care (e.g., public and private insurance coverage eligibility, usual source of care, unmet need, etc.), diagnoses, social history, family history, medical visit data, radiology, procedures, lab values, lab trends, lab deltas, medications, follow up, genetic markers, exercise habits, shopping habits, patient survey answers, diet, disabilities and impairments, mental health history, addiction treatment history, medical expense data, and other clinical and lifestyle information. In some embodiments, the healthcare data received at act 402 is specific to a particular condition or set of conditions (e.g., co-morbidities). For example, for diabetes patients, the healthcare data may include glucose screening and control history, lipid screening and control history, blood pressure screening and control history, nephropathy screening and control history, eye and foot care history, history of preventative care, hospital inpatient claims, number and types of medications, history of preventative care, and claims data (e.g., claims for immunization and procedures.)

In some embodiments, receiving the healthcare data at act 402 includes receiving the healthcare data from one or more users. For example, the user(s) may include a Provider, a patient, and/or any other suitable entity. In some embodiments, the healthcare data 402 is received in response to the user uploading a file including the healthcare data. In some embodiments, the healthcare data 402 is received in response to the user entering the healthcare data via a user interface of a computing device (e.g., user interface module 234 shown in FIG. 2).

In some embodiments, receiving the healthcare data at act 402 includes obtaining a user's response to a prompt generated by a machine learning model. For example, a machine learning model, such as a large language model, may be configured to generate prompts requesting data from a user. In some embodiments, the requested data includes missing data needed for determining the care gap score. In some embodiments, the prompts are responsive to user input and/or existing healthcare data. For example, the machine learning model may process the user input and/or existing healthcare data and generate prompts that include follow-up questions for obtaining additional information from the user, thereby generating a more comprehensive set of data for determining the care gap score. Examples of machine learning models are described herein including at least in the section “Machine Learning.”

At act 404, a rule is applied to the healthcare data obtained for the patient to determine whether the patient has a care gap associated with the rule. For example, a rule may be used to distinguish between patients having the care gap versus patients who do not have the care gap. For example, a rule may specify an action and/or condition. Patients who have not taken the action and/or have the condition may be identified as having a care gap, while patients who have taken the action and/or do not have the condition may be identified as lacking the care gap. Alternatively, patients who have taken the action and/or do not have the condition may be identified as having a care gap, while patients who have not taken the action and/or have the condition may be identified as lacking the care gap. As a nonlimiting example, consider a rule specifying “diabetes patient with at least 1 inpatient encounter over the past two years.” Applying the rule to the patient's healthcare data may involve analyzing diagnoses data and/or medical data to determine whether the patient is a diabetes patient who has had at least 1 inpatient encounter over the past 2 years. In this example, if the patient is a diabetic patient who has had at least 1 inpatient encounter over the past 2 years, then the patient may be identified as having a care gap. If not, then the patient may be identified as not having a care gap.

At act 406, an individual score is provided for the care gap associated with the rule. In some embodiments, different scores are provided for the care gap depending on whether the patient was determined to have the care gap. Consider for example, a care gap associated with the rule “diabetes patient with at least 1 inpatient encounter over the past two years.” If the patient is determined to have the care gap as a result of applying the rule (e.g., they are a diabetic with at least 1 inpatient encounter over the past two years) then a first score (e.g., a score of +1, or any other suitable value) may be provided for the care gap. If the patient is identified as not having the care gap as a result of applying the rule (e.g., they are not a diabetic with at least one inpatient encounter over the past two years), then a second score (e.g., a score of 0, or any other suitable value) may be provided for the care gap different from the first score.

At act 408, in some embodiments, a weight may optionally be applied to the individual score provided for the care gap. In such embodiments, the weight may depend on the type of care gap. For example, a greater weight may be applied to a care gap that poses a greater opportunity to improve patient health. For example, a care gap may exist for a patient who smokes; there is an opportunity to decrease their risk of lung cancer by quitting smoking. Addressing this care gap may have a more significant impact on patient health than a care gap that exists because the patient exercises only twice a week, instead of five times a week. Accordingly, a greater weight may be assigned to the individual score for the smoking care gap than for the exercise care gap.

In some embodiments, care gaps are grouped to form a care gap group. In some embodiments, a care gap group covers a specific set of patients or patient profiles. For example, a care gap group may include care gaps associated with a particular diagnosis such as diabetes. In some embodiments, each care gap in the care gap group is associated with a rule for determining whether the patient has that particular care cap. For example, a care gap group for diabetic patients may include care gaps associated with the following rules to be applied to a healthcare data for a diabetic patient: (a) at least 2 outpatient encounters with a diagnosis of diabetes over two years; (b) at least 1 inpatient encounter over 2 years; (c) prescription for diabetes medications other than Metformin, and (d) HbA1c is available and >6.5%. Accordingly, in some embodiments, act 410 includes determining whether there is another rule associated with another care gap (e.g., in a care gap group). For example, if rule (a) was applied to the healthcare data at act 404, then act 410 may include determining that there is at least one other rule (e.g., rules (b), (c), and (d)) associated with care gaps included in the example care gap group for diabetic patients.

If, at act 410, it is determined that there is another rule is associated with a care gap (e.g., another rule associated with a care gap in a care gap group), then acts 404-408 may be repeated for the rule. For example, the rule may be applied to the patient's healthcare data to determine whether the patient has the care gap, an individual score may be provided for the care gap based on a result of the determination, and a weight may optionally be applied to the individual score provided for the care gap.

If, at act 410, there are no further rule associated with a care gap, then process 400 proceeds to act 412. At act 412, a care gap score is determined based on the individual score(s) provided to the care gap(s) at act 406 and the weights applied at act 408. In some embodiments, this includes determining a linear and/or non-linear combination of the individual scores. In such an embodiment, when weights are applied at act 408, then the linear and/or non-linear combination is weighted based on the weights applied to the individual scores.

In some embodiments, the care gap score determined at act 412 is a single value. For example, the single value may represent the care gap score of a care group described with respect to act 410.

In some embodiments, the care gap score is normalized to a standard scale. For example, the care gap score may be normalized to a scale ranging from 0 to 20, with 20 representing the highest care gap score and 0 representing the lowest care gap score. However, it should be appreciated that any suitable scale may be used to normalize the care gap scores. Examples of normalizing the care gap score are described herein including at least with respect to FIG. 3B.

Risk Score

Aspects of the technology described herein relate to determining a risk score for a patient. In some embodiments, the risk score represents a patient's likelihood of having adverse events.

In some embodiments, risk is determined using any suitable risk score model. Non-limiting examples of risk score models include Persivia's Soliton Suggested Risk Score (SSR) model, the Social Determinants of Health (SDOH) risk score, and the American Academy of Family Physicians (AAFP) risk stratification score. However, any other suitable risk score model may be used to predict a risk score for the patient, as aspects of the technology described herein are not limited in this respect.

In some embodiments, the risk score is determined based on the patient's healthcare data. As nonlimiting examples, the risk score determination module 224 may be configured to process data including demographic data (e.g., age, sex race, ethnicity, nationality, nativity/time in a country, language, etc.), socioeconomic data (e.g., household income, education, employment, food insecurity, housing insecurity, crime prevalence, transportation issues, household composition (e.g., whether a patient lives alone), etc.), attitudes and values (e.g., risk tolerance, attitudes about insurance and doctors, past experiences with healthcare providers, etc.), community data (e.g., urban-rural location of residence, point of care, etc.), access to care (e.g., public and private insurance coverage eligibility, usual source of care, unmet need, etc.), diagnoses, social history, family history, medical visit data, radiology, procedures, lab values, lab trends, lab deltas, medications, follow up, genetic markers, exercise habits, shopping habits, patient survey answers, diet, disabilities and impairments, mental health history, addiction treatment history, medical expense data, and other clinical and lifestyle information.

FIG. 12 is an example table used for determining a risk score according to Persivia's SSR model. A “chronic condition” in the table refers to: cancer of any kind (including colorectal cancer survivor); acquired hypothyroidism; allergic rhinitis; anxiety; asthma; bilateral knee pain; bursitis tendinitis; chronic back pain; chronic hemolytic anemia; gout; gerd; knee pain; and muscle spasm.

In the example of FIG. 12, the rules listed in row 1202 may be used to determine a risk score for the patient. As one example, a risk score of 2 is identified for patients with an HBA1c greater than 6.5 and a BMI greater than or equal to 18.5 and less than or equal to 24.9. As shown, a risk score of 2 is considered “low risk.”

Row 1204 identifies care plan considerations for the patient based on the determined risk score. For example, the care plan consideration for a risk score of 2 include basic intervention, online and/or patient education, and monitoring. In some embodiments, the care plan considerations may be displayed via a GUI (e.g., the GUI generated according to process 1400 in FIG. 14).

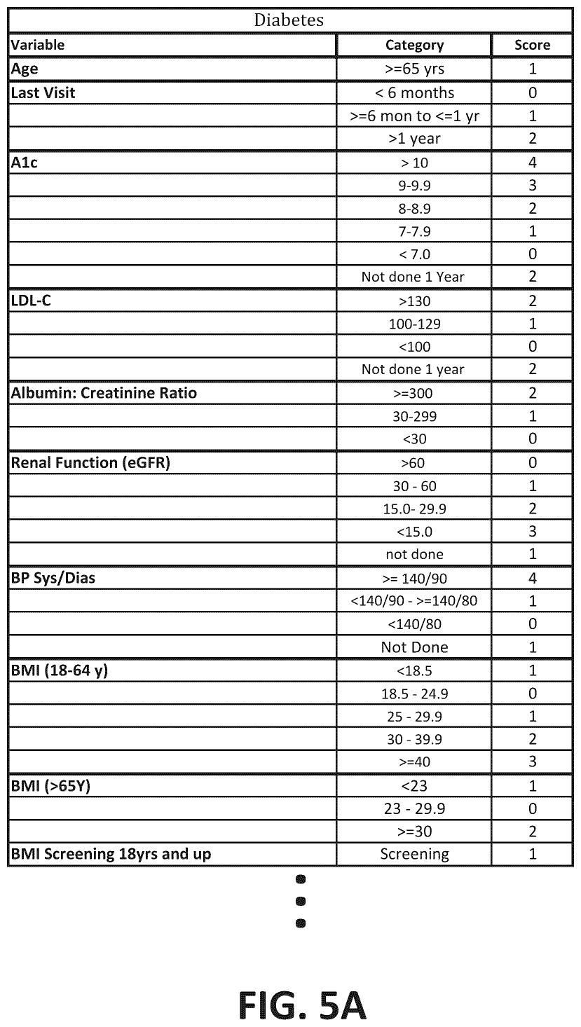

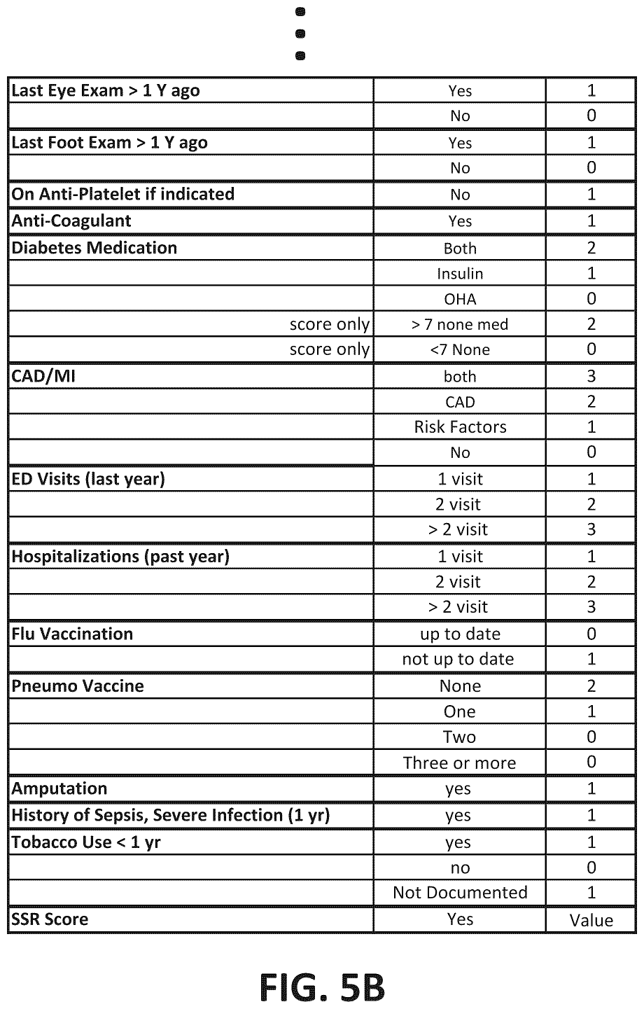

In some embodiments, the healthcare data used to determine the risk score depends on a diagnosis of the patient. The examples shown in FIGS. 5A-11B identify different types of healthcare data used to determine risk scores for different diagnoses. These examples do not include real patient data. While various examples of determining risk scores for particular patients with particular diagnoses are described herein, it should be appreciated that a risk score may be determined for any patient with any diagnoses, conditions, or combinations thereof, as aspects of the technology described herein are not limited in this respect.

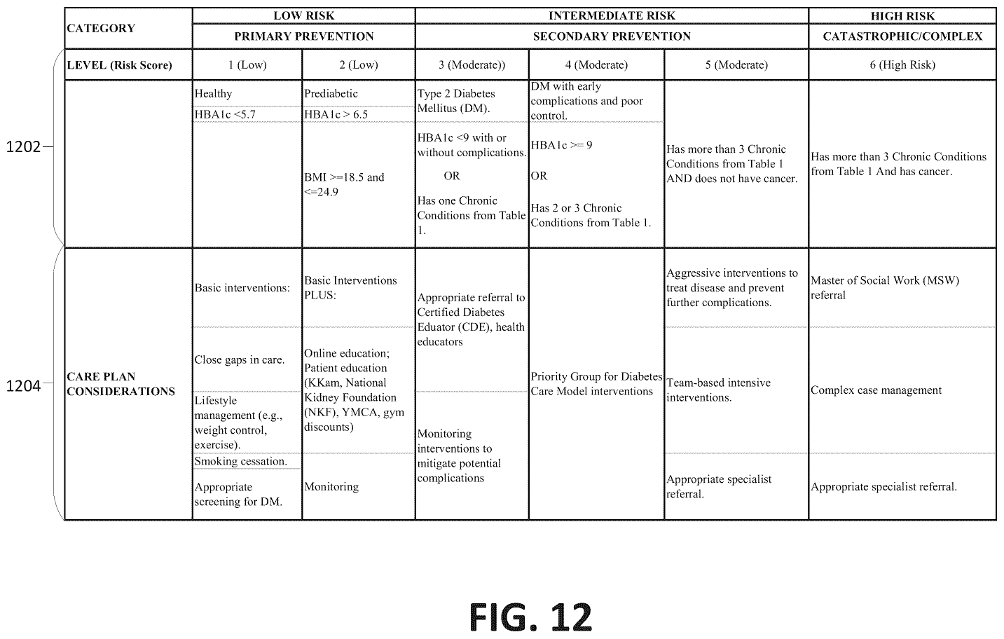

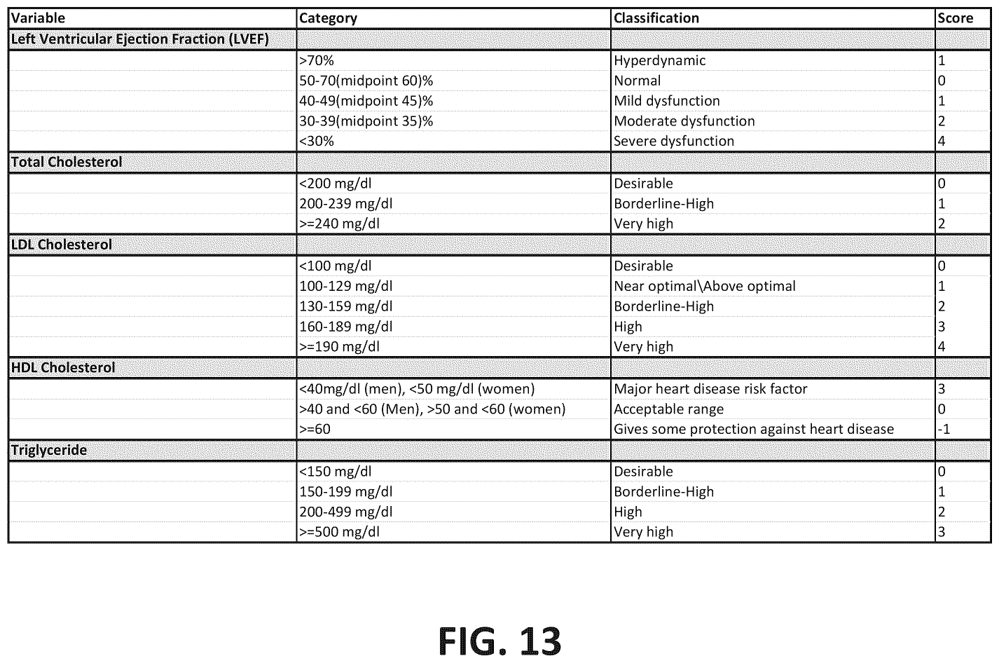

FIG. 13 shows examples of different variables used to determine the risk scores shown in the examples of FIGS. 5A-11B. Ranges of values for each variable are provided. Each range of values is associated with a score used in determining the example risk scores.

FIG. 5A shows rows of and FIG. 5B shows further rows of an exemplary table for determining a risk score for diabetes.

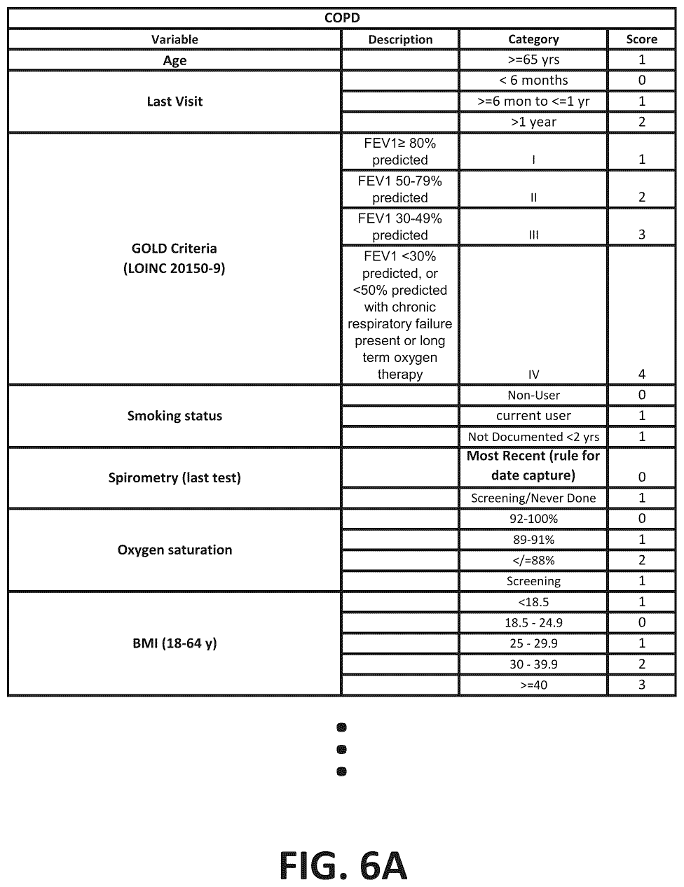

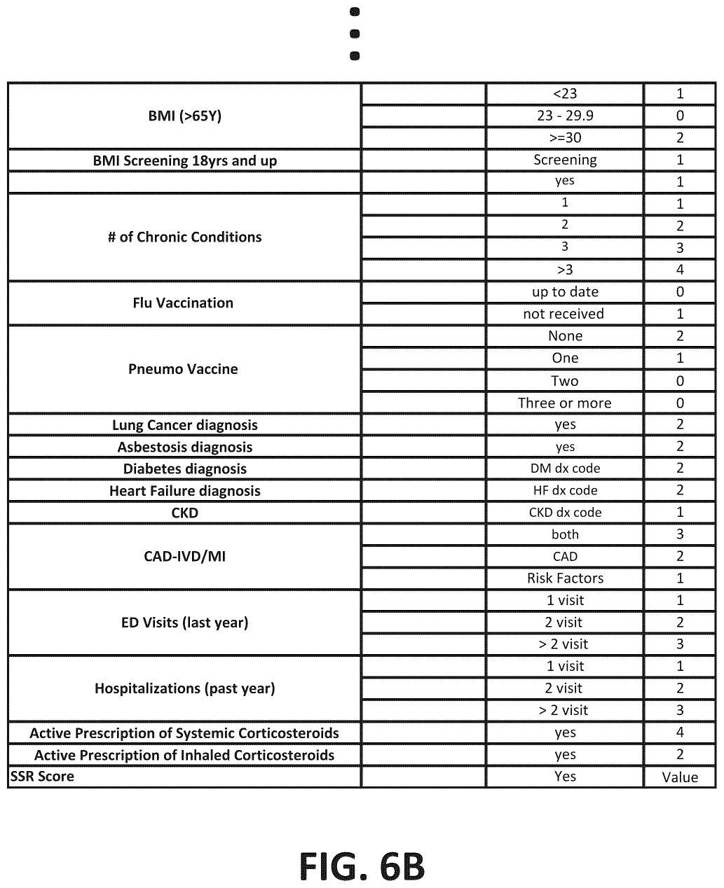

FIG. 6A shows rows of and FIG. 6B shows further rows of an exemplary table for determining a risk score for chronic obstructive pulmonary disease (COPD).

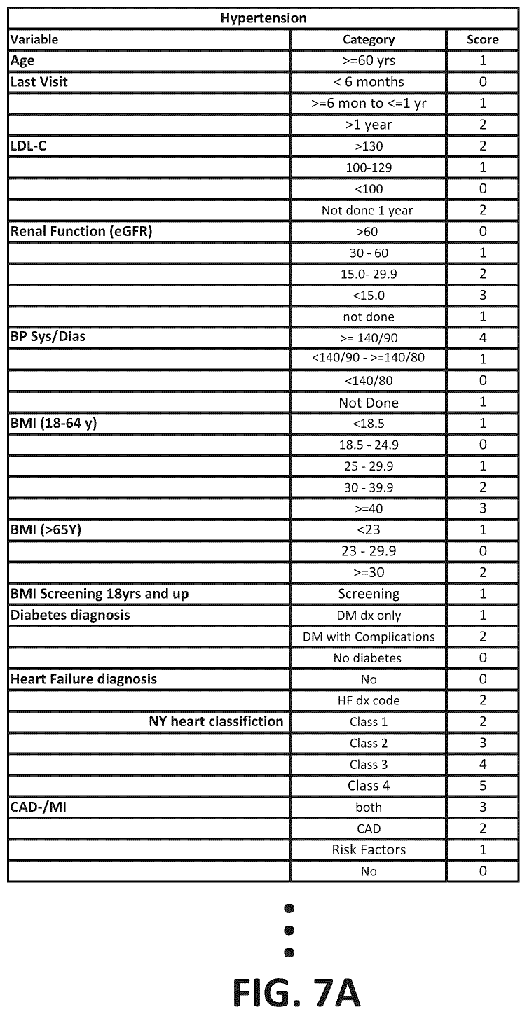

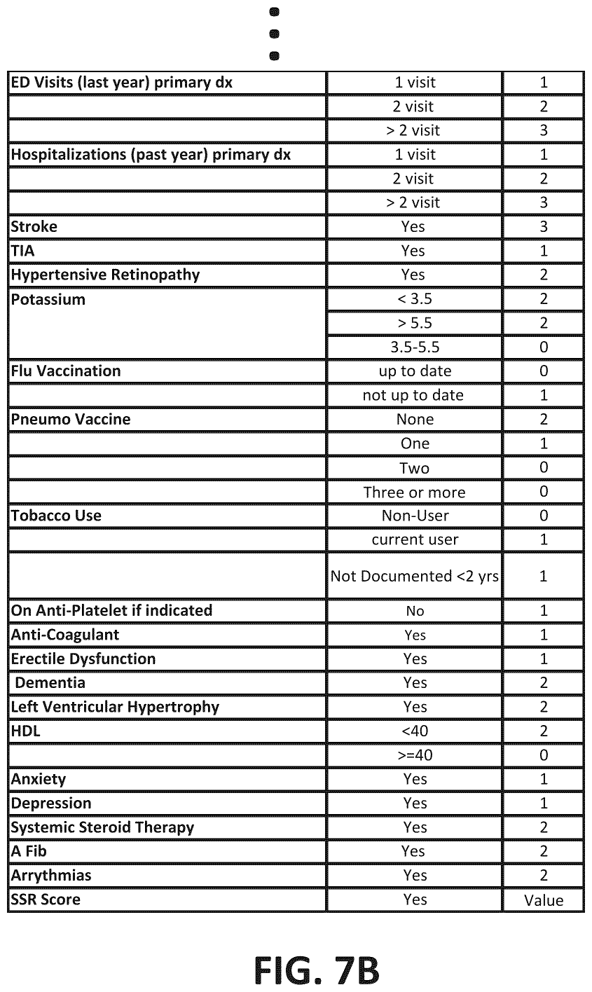

FIG. 7A shows rows of and FIG. 7B shows further rows of an exemplary table for determining a risk score for hypertension.

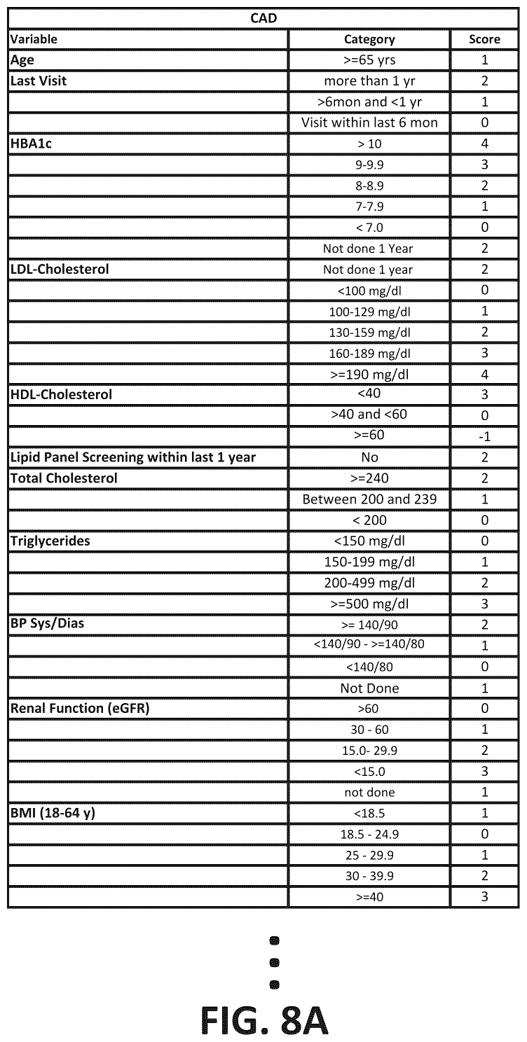

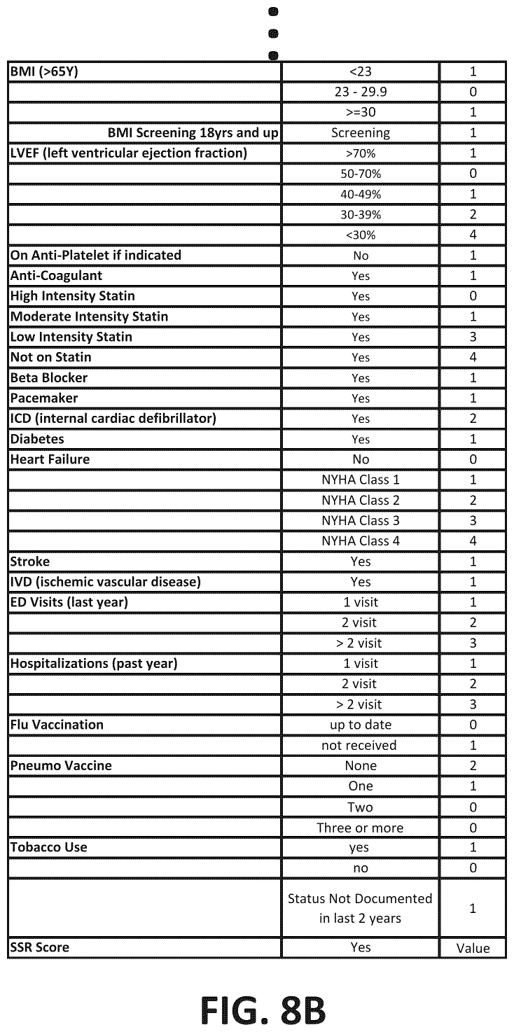

FIG. 8A shows rows of and FIG. 8B shows further rows of an exemplary table for determining a risk score for coronary artery disease (CAD).

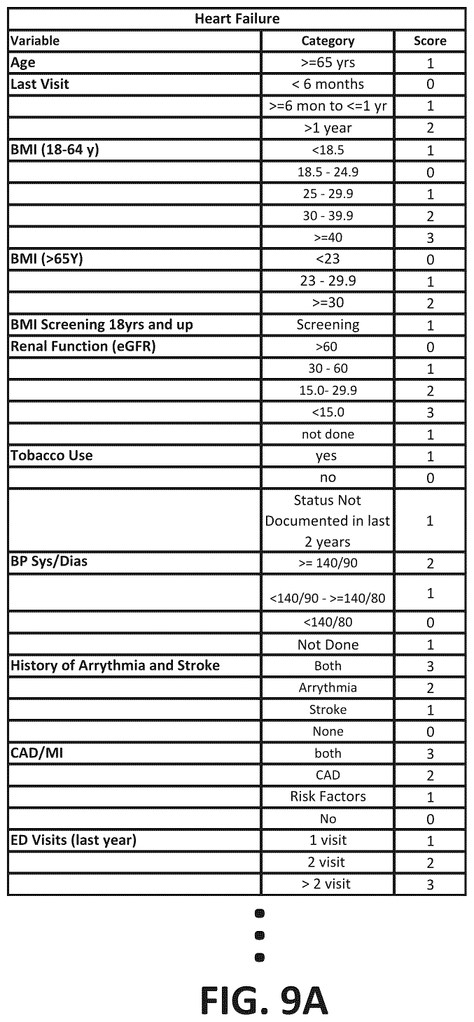

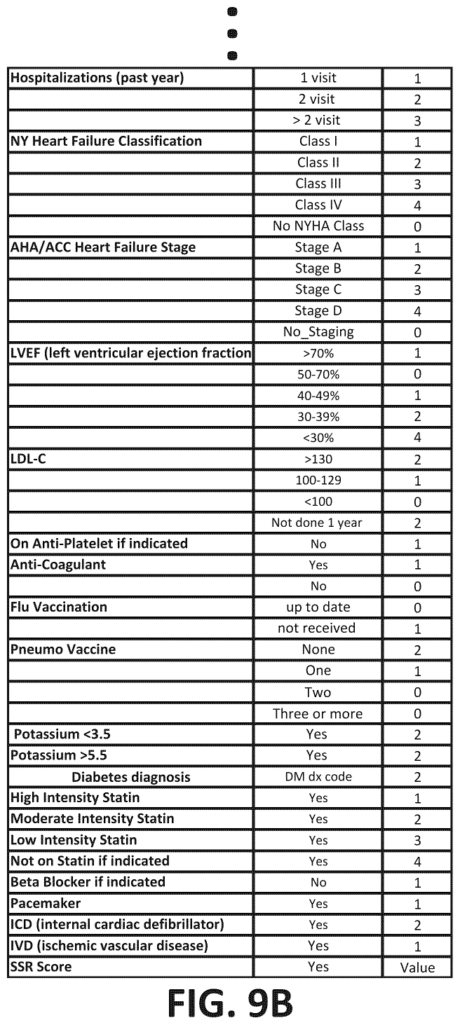

FIG. 9A shows rows of and FIG. 9B shows further rows of an exemplary table for determining a risk score for heart failure.

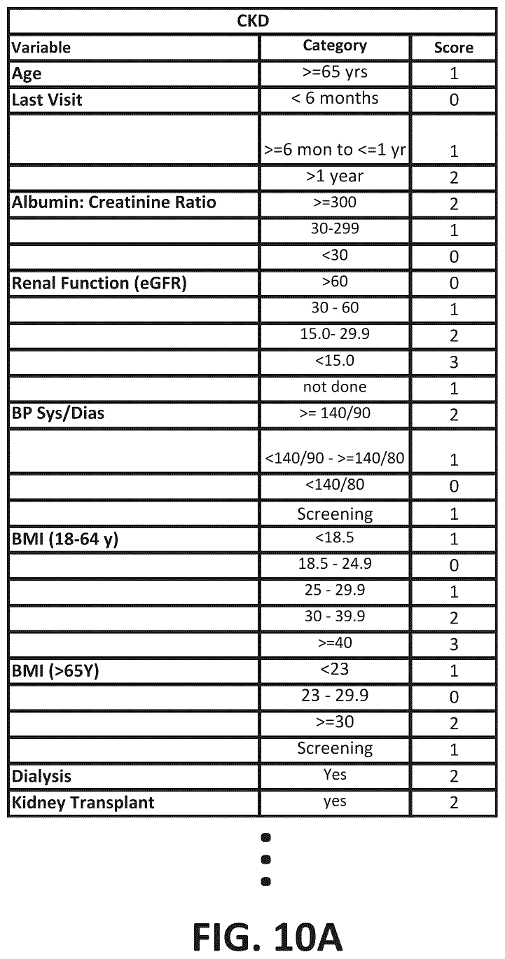

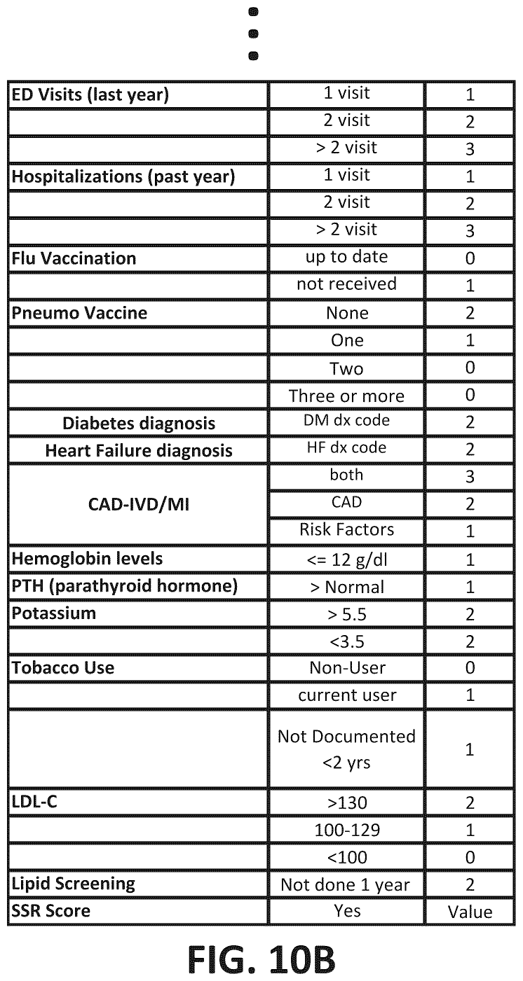

FIG. 10A shows rows of and FIG. 10B shows further rows of an exemplary table for determining a risk score for chronic kidney disease (CKD).

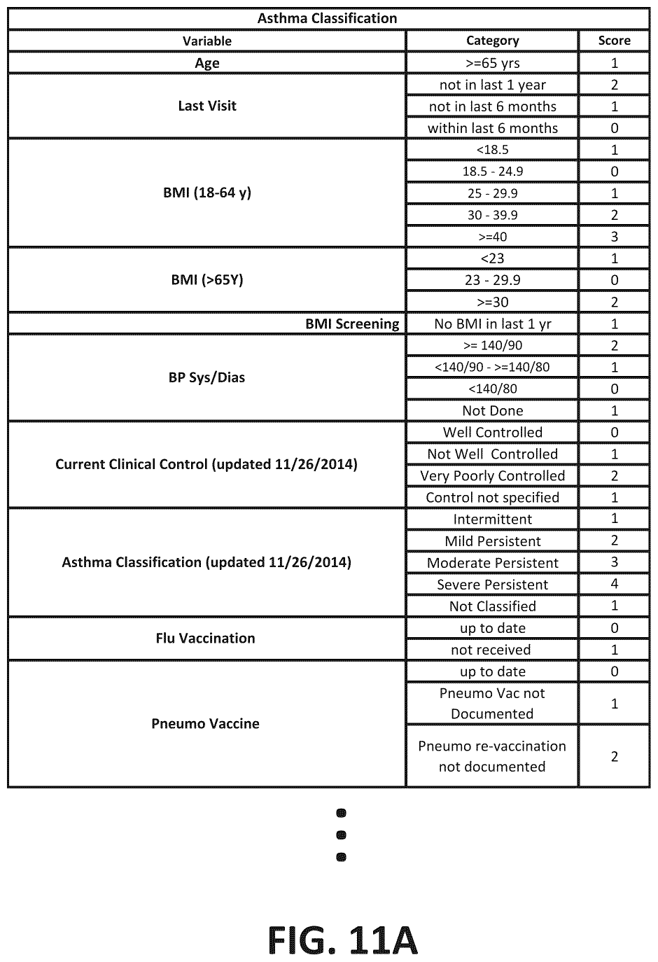

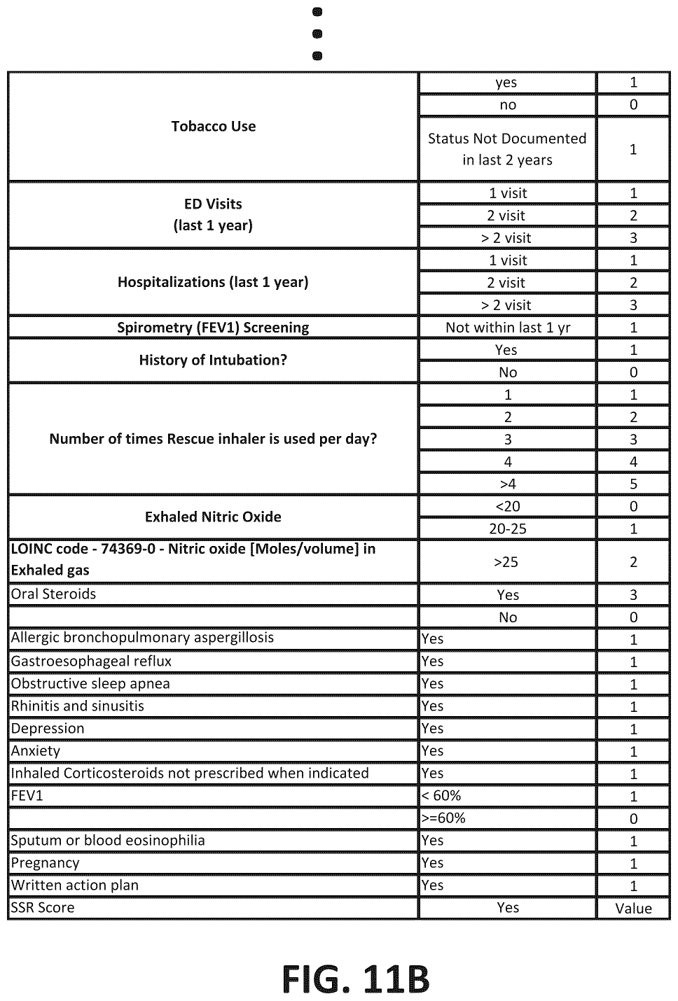

FIG. 11A shows rows of and FIG. 11B shows further rows of an exemplary table for determining a risk score for asthma.

Change in Risk Score

Aspects of the technology described herein relate to determining a change in risk score for a patient.

In some embodiments, multiple (e.g., 2 or more) risk scores are obtained for a patient over a time period. For example, a first risk score may be obtained at a first time point and a second risk score (e.g., a current risk score) may be obtained at a second time point after the first time point (e.g., hours, days, weeks, months, or years after the first time point).

In some embodiments, a change in risk score is determined based on the multiple risk scores. For example, the change in risk score may reflect a percent change in risk score, a difference between two risk scores, a slope of a line fitted to multiple risk scores over time, or any other suitable measure of change, as aspects of the technology described herein are not limited in this respect.

Patient Willingness

Aspects of the technology described herein relate to determining a measure representing patient willingness. In some embodiments, the measure may represent a patient's willingness to follow instructions and/or to perform activities that are beneficial to their health. As non-limiting examples, the measure may reflect patient willingness to comply with a medication dosage schedule, responsiveness to communications from providers, willingness to receive routine medical examinations, willingness to undergo certain procedures, reception to preventative care (e.g., vaccines), and willingness to visit specialists.

In some embodiments, the measure of patient willingness is determined based on the patient's answers to one or more surveys regarding the patient's attitude toward taking charge of their own treatment. For example, the answers may indicate whether the patient agrees or disagrees with statements included in the survey. Nonlimiting examples of such statements include: (a) I understand that I am responsible for managing my health; (b) I understand what factors impact my health; (c) I understand the nature of my health conditions; (d) I know what medications I am taking; (d) I understand what my medications are supposed to do for me.

In some embodiments, each of the one or more surveys may include any suitable number of statements and/or questions to which the patient can provide response(s), as aspects of the technology described herein are not limited in this respect. For example, each survey may include fewer than 30, fewer than 25, fewer than 20, fewer than 15, fewer than 10, or fewer than 5 statements and/or questions.

In some embodiments, each statement and/or question has a numerical score associated with each of the various possible responses. Consider, for example, the statement: “I understand that I responsible for managing my health.” One numerical score (e.g., 1) may be associated with a response agreeing with that statement, while a different numerical score (e.g., 0) may be associated with a response disagreeing with that statement.

In some embodiments, the numerical scores are used to determine the final measure representing patient willingness. The numerical scores may be combined in any suitable manner to determine the final measure. For example, the measure may be obtained by determining a weighted or unweighted sum of the scores.

In some embodiments, the patient's responses are provided by the patient. For example, the patient may provide the responses via a user interface (e.g., a GUI), upload the responses, provide instructions to a device used to transmit the responses, or provide the responses in any other suitable manner, as aspects of the technology are not limited in this respect. In some embodiments, the patient may provide the responses to a Provider or other entity who may then enter or otherwise provide the responses to the computing device used to process the responses.

In some embodiments, the patient may respond to a prompt generated by a machine learning model. For example, a machine learning model, such as a large language model, may be configured to generate prompts requesting answers and/or responses to survey questions. In some embodiments, the prompts are responsive to user input and/or existing healthcare data. For example, the machine learning model may process the user input and/or existing healthcare data and generate prompts that are specific to the user. Examples of machine learning models are described herein including at least in the section “Machine Learning.”

Prediction of Future Cost(s)

Aspects of the technology described herein relate to determining a prediction of future cost(s) associated with care of a patient.

In some embodiments, future cost(s) may be predicted based on the patient's healthcare data. As nonlimiting examples, the future cost(s) may be predicted based on claims data (e.g., data used to process insurance claims) such as medical visit data, diagnoses, treatments, procedures, medications, and/or any other suitable healthcare data for the patient.

In some embodiments, any suitable cost prediction model may be used to process the healthcare data to obtain the prediction of future costs. Non-limiting examples of cost risk prediction models include the Centers for Medicare and Medicaid Services Hierarchical Condition Category (CMS-HCC) risk adjustment model and the Johns Hopkins ACG System. However, any other suitable cost risk prediction model may be used to predict future cost(s) for the patient, as aspects of the technology described herein are not limited in this respect.

In some embodiments, a machine learning model may be used to predict future cost(s) and/or a cost range for a patient based on characteristics of the patient. For example, the machine learning model may be configured to process data such as patient gender and age, for example, to obtain an output indicative of the predicted future costs associated with care of that patient. In some embodiments, the machine learning model may include a decision tree classifier, a gradient boosted decision tree classifier, a neural network, a support vector machine classifier, or any other suitable type of machine learning model, as aspects of the technology described herein are not limited in this respect. In some embodiments, the machine learning model may include an ensemble of machine learning models of any suitable type (the machine learning models part of the ensemble may be termed “weak learners”). For example, the machine learning model may include an ensemble of decision tree classifiers, such as a random forest classifier (e.g., as described, for example, in Breiman, L. (2001). Random forests. Machine learning 45 (2001): 5-32).

As a non-limiting example, a random forest classifier was trained to predict future cost(s) for a patient based on patient healthcare data. In particular, the example model was trained to classify the patient into one of two classes: 1 and 0. 1 represents future costs greater than or equal to $11,000 and 0 represents future costs of less than $11,000.

In the example, the random forest classifier was trained using patient data and the costs associated with providing care for those patients. Both the patient data and the cost data were pre-processed prior to using it to train the random forest classifier.

Pre-processing the patient data included performing categorization for patient data such as gender and age. With respect to gender, females were assigned a value of 1 and males were assigned a value of 2. With respect to age, patients were divided into two categories: patients under 65 were assigned a value of 0 and patients 65 and older were assigned a value of 2.

Pre-processing the cost data included determining a 12-month cost for each patient. The 12-month cost is determined based on costs accrued during a length of stay. Because the patients' length of stay varied, so did the costs available for determining the 12-month costs. For example, sometimes only 1 month's cost is available for a patient, while other times, 12 months' cost is available. To account for this variability, the 12-month cost for each patient was determined by determining 1 month's cost multiplied by 12.

The data was split into training data and testing data and used to train and test the random forest classifier, respectively. The performance of the random forest classifier is shown in Tables 1-1 and 1-2, below.

TABLE 1-1
Performance of random forest classifier in predicting patient costs.

| | Cost | Total Cost | Patient | | | |
|---|---|---|---|---|---|---|
| Buckets | Ranges | in Buckets | Count | Percentage | 1 | 0 |
| 1 | >=$11k | $64,614,297.65 | 506 | 30.2 | 427 | 79 |
| 0 | <$11k | $4,980,472.56 | 1170 | 69.8 | 76 | 1094 |

TABLE 1-2
Performance of random forest classifier in predicting patient costs.

| | | | Negative | | |
|---|---|---|---|---|---|
| | Recall/ | | Predictive | Accuracy | Error Rate |
| Precision | Sensitivity | Specificity | Value | TP + TN/ | FN + FP/ |
| TP/TP + | TP/TP + | TN/TN + | TN/TN + | TP + FP + | TP + FP + |
| FP | FN | FP | FN | FN + TN | FN + TN |
| 0.85 | 0.84 | 0.94 | 0.93 | 0.91 | 0.09 |

In some embodiments, the techniques described herein may be used to determine how to prioritize patients in administering healthcare. The techniques may be used to both rank patients with respect to one another (i.e., to determine which patients to prioritize), in addition to keeping track of healthcare records for each patient for efficiently providing healthcare services to each patient. Accordingly, the techniques provide for an efficient network-based patient management system that collects, consolidates, and processes patient information from various Providers into a standardized format (e.g., standardized to the PPS scale/ranking), stores it in network-based storage devices (e.g., healthcare data store 270 in FIG. 2), and generates prompts notifying Providers of the level of care to be administered to a patient once the patient information has been processed and/or compared to patient information associated with other patients (e.g., comparing PPSs across various patients).

In some embodiments, the techniques developed by the inventors include generating a graphical user interface (GUI) by a computing device (e.g., computing device(s) 210 shown in FIG. 2), which is hardware or a combination of both hardware and software (e.g., software 250, including user interface module 234 shown in FIG. 2). In some embodiments, a user, such as a Provider, patient, or other suitable entity is given access (e.g., remote access) through the GUI to view or update information about the patient's medical condition using a device (e.g., the user's own local device, such as a personal computer or wireless handheld device.) In some embodiments, when the user wants to update the record, the user can input the update in any format used by the user's local device. In some embodiments, when the patient information is updated, it may be converted to a suitable format and then stored in the medical records on the network-based storage device (e.g., healthcare data store 270 in FIG. 2). In some embodiments, the updated information may be processed and used to determine a new or updated PPS for the patient. The PPS may be used to generate a GUI that includes a prompt to the Provider to administer a level of care to the patient. Additionally, or alternatively, in some embodiments, the PPS may be used to generate multiple different GUIs, each of which includes a respective prompt to be displayed to a respective Provider to administer a respective level of care to one or more other patients. For example, patient information may be updated for one patient. The patient information may be used to determine an updated PPS for that patient. As a result, the PPS for one or more other patients may be updated. For example, if the updated patient information results in an increased PPS for the patient, this may indicate that the patient should be prioritized over other patients. A GUI may be generated to indicate, to the patients' Provider(s) that their PPSs have been updated and that they should be treated with lower priority.

FIG. 14A is a flowchart of an illustrative process for generating a graphical user interface (GUI) including a prompt to a healthcare professional to administer a level of care to the patient, according to some embodiments of the technology described herein. Process 1400 may be performed by software (e.g., user interface module 234 in FIG. 2) executing on a laptop computer, a desktop computer, one or more servers, in a cloud computing environment, computing device 106 described with respect to FIG. 1A, computing device 210 described with respect to FIG. 2, computing device 1500 described with respect to FIG. 15, or any other suitable computing device.

At act 1402, a GUI is generated that includes a prompt to a healthcare professional to administer a level of care to a patient. In some embodiments, the prompt is determined based on the PPS determined for the patient. For example, the GUI may indicate the value of the PPS determined for the patient. Additionally, or alternatively, in some embodiments, the GUI may indicate a ranking of patients based on the PPS s determined for the patients. Additionally, or alternatively, in some embodiments, the GUI may include one or more recommended actions for treating the patient. For example, depending on the PPS, the GUI may include recommendations for interventions such as lifestyle management, preventative screenings, monitoring, specialist referrals, patient education, complex case management, or any other suitable recommendations, as aspects of the technology described herein are not limited in this respect.

At act 1404, the GUI is provided to a display. For example, the GUI may be provided to the display of any suitable computing device such as, for example, the display of a personal computer, handheld device (e.g., a smartphone), or any other suitable device, as aspects of the technology described herein are not limited in this respect.

FIG. 14B is an example GUI 1450 including a prompt to a healthcare professional to administer a level of care to a patient, according to some embodiments of the technology described herein. The example GUI 1450 does not include real patient data.

As shown, the example GUI includes patient information including name and age. The example GUI also shows the PPS determined for each patient, in addition to values determined for factors used to determine the PPS (e.g., risk score, change in risk score, cost risk, patient activation measure (measure representing patient willingness)).

In some embodiments, the PPS for each patient may indicate to the Provider what level of care should be provided to a particular patient. In the example, Patient A has a lower PPS than Patient B. This may indicate to a Provider that a lower level of care should be provided to Patient A relative to the care provided to Patient B.

In some embodiments, a user may interact with the GUI. For example, the GUI includes buttons 1452. By selecting the buttons, the user may select the information that is displayed to the user and/or interact with the information currently displayed. For example, the user may select the “Messaging” tab to display a GUI configured for messaging. As another example, the user may select the “+Patient” tab to add a patient to the list shown in the current GUI.

It should be appreciated that the GUI may include any suitable GUI and is not limited by the example GUI shown in FIG. 14B.

Machine Learning

According to some embodiments of the technology described herein, a machine learning model is used to generate prompts for obtaining user input. For example, as described herein including at least with respect to FIG. 2, FIG. 3A, and the section “Care Gap Score,” a machine learning model may be used to identify and/or collect clinically-relevant data from a patient. As another example, as described herein including at least with respect to FIG. 2, FIG. 3A, and the section “Patient Willingness,” a machine learning model may be used to generate prompts for users to provide responses to statements and/or questions used for measuring patient willingness.

The machine learning model may include any suitable machine learning model, as embodiments of the technology described herein are not limited in this respect. For example, the machine learning model may include any suitable large language model (LLM). Nonlimiting examples of LLMs that may be used to implement the techniques described herein include GPT-4, Falcon-40B, LAION Open Assistance, Nomic.ai Gpt4all, and Databricks Dolly 2.0. GPT-4 is described by OpenAI, R. (“GPT-4 technical report.” arXiv (2023): 2303-08774), which is incorporated by reference herein in its entirety. Falcon-40B is a causal decoder-only that was developed by Technology Innovation Institute. Nomic.ai Gpt4all is described by Yuvanesh, A. et al. (“GPT4A11: Training an Assistant-style Chatbot with Large Scale Data Distillation from GPT-3.5-Turbo.” GitHub repository (2023)”), which is incorporated by reference herein in its entirety. Databricks Dolly 2.0 is described by Conover, M. et al. (“Free Dolly: Introducing the world's first truly open instruction-tuned LLM. Databricks. (2023)), which is incorporated by reference herein in its entirety. Additionally, training language models to follow instructions with human feedback is described by Ouyang, L. et al. (“Training language models to follow instructions with human feedback.” Advances in Neural Information Processing Systems 35 (2022): 27730-27744), which is incorporated by reference herein in its entirety.

Computer Implementation

An illustrative implementation of a computer system 1500 that may be used in connection with any of the embodiments of the disclosure provided herein is shown in FIG. 15. The computer system 1500 may include one or more processors 1502 and one or more articles of manufacture that comprise non-transitory computer-readable storage media, for example, memory 1504 and one or more non-volatile storage media 1506. The processor 1502 may control writing data to and reading data from the memory 1504 and the non-volatile storage device 1506 in any suitable manner. To perform any of the functionality or methods described herein, for example, process 300, process 400, or process 1400, the processor 1502 may execute one or more processor-executable instructions stored in one or more non-transitory computer-readable storage media, for example the memory 1504, which may serve as non-transitory computer-readable storage media storing processor-executable instructions for execution by the processor 1502.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

The terms “approximately,” “substantially,” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

In view of the described embodiments of the techniques described herein and variations thereof, below are described certain more particularly described aspects. These particularly recited aspects should not however be interpreted to have any limiting effect on any different claims containing different or more general teachings described herein, or that the “particular” aspects are somehow limited in some way other than the inherent meanings of the language literally used therein.

1. A method of determining a patient prioritization score of each particular patient of a plurality of patients for use by a healthcare professional in administering care to the plurality of patients, the method comprising:
receiving healthcare data for each particular patient of the plurality of patients;
determining, based on the healthcare data for each particular patient, a respective patient prioritization score for each particular patient of the plurality of patients, the determining comprising:
determining a care gap score for the particular patient based on a first subset of the healthcare data associated with the particular patient, the care gap score representing opportunities to improve care of the patient;
determining a risk score for the particular patient based on a second subset of the healthcare data associated with the particular patient, the risk score representing risks to health of the patient;
determining a change over a time period in the risk score for the particular patient;
determining a measure representing a willingness of the particular patient to receive care;
determining a prediction of future costs associated with care of the particular patient
determining the patient prioritization score for the particular patient based on the care gap score, the risk score, the change over the time period, and the prediction of the future costs;
determining, based on the respective patient prioritization score determined for each of the plurality of patients, a respective level of care to be administered to each particular patient of the plurality of patients; and
prompting the healthcare professional to administer the determined respective level of care to each particular patient of the plurality of patients.

2. The method of claim 1, wherein the plurality of patients include at least 2 patients, at least 5 patients, at least 10 patients, at least 25 patients, at least 50 patients, at least 100 patients, at least 500 patients, at least 1,000 patients, or at least 5,000 patients.

3. The method of claim 1, wherein determining the respective level of care to be administered to each particular patient of the plurality of patients comprises:
determining that more resources should be allocated to patients of the plurality of patients for whom a higher patient prioritization score was determined than patients for whom a lower patient prioritization score was determined.

4. The method of claim 1 wherein determining a care gap score for the particular patient based on the first subset of healthcare data for the particular patient comprises:
applying a rule to the healthcare data for the particular patient, wherein the rule is associated with a care gap;
determining, based on a result of applying the rule, whether the particular patient has the care gap associated with the rule;
providing an individual score for the care gap based on a result of determining whether the particular patient has the care gap;
applying a weight to the individual score for the care gap; and
determining the care gap score based on the weight and the individual score.

5. The method of claim 4, wherein:
the first subset of the healthcare data associated with the particular patient comprises at least one of demographics, diagnosis, social history, family history, medical visit data, radiology, procedures, lab values, lab trends, lab deltas, medication, follow ups, genetic markers, exercise habits, and shopping habits; and
applying the rule to the first subset of healthcare data for the particular patient comprises applying the rule to the at least one of demographics, diagnosis, social history, family history, medical visit data, radiology, procedures, lab values, lab trends, lab deltas, medication, follow up, genetic markers, exercise habits, and shopping habits.

6. The method of claim 1, wherein receiving the healthcare data for each patient of the plurality of patients comprises:
using a large language model to generate a respective prompt requesting input from each particular patient of the plurality of patients; and
receiving at least some of the healthcare data in response to generating the respective prompt requesting input from each particular patient of the plurality of patients.

7. A method of determining a patient prioritization score of a patient for use by a healthcare professional in administering care to the patient, the method comprising:
receiving healthcare data associated with the patient;
determining, based on the healthcare data for the patient, a care gap score for the patient representing opportunities to improve care of the patient;
determining, based on the healthcare data for the patient, a risk score for the patient representing risks to health of the patient;
determining, based on the care gap score and the risk score, the patient prioritization score of the patient;
prompting, based on the patient prioritization score for the patient, the healthcare professional to administer a level of care to the patient.

8. The method of claim 7 wherein determining a care gap score for the patient based on the healthcare data for the patient comprises:
applying a rule to the healthcare data for the patient, wherein the rule is associated with a care gap;
determining, based on a result of applying the rule, whether the patient has the care gap associated with the rule;
providing an individual score for the care gap based on a result of determining whether the patient has the care gap;
applying a weight to the individual score for the care gap; and
determining the care gap score based on the weight and the individual score.

9. The method of claim 8, wherein:
the healthcare data associated with the patient comprises at least one of demographics, diagnosis, social history, family history, medical visit data, radiology, procedures, lab values, lab trends, lab deltas, medication, follow ups, genetic markers, exercise habits, and shopping habits; and
applying the rule to the healthcare data for the patient comprises applying the rule to the at least one of demographics, diagnosis, social history, family history, medical visit data, radiology, procedures, lab values, lab trends, lab deltas, medication, follow up, genetic markers, exercise habits, and shopping habits.

10. The method of claim 7, wherein:
the healthcare data associated with the patient comprises at least one of clinical data, claims data, event data, device data, patient reported data or socio-economic data; and
determining a risk score for the patient based on the healthcare data for the patient comprises determining the risk score for the patient based on the at least one of the clinical data, claims data, event data, device data, patient reported data or socio-economic data.

11. The method of claim 7, further comprising:
determining a change over a time period in the risk score for the patient; and
determining the patient prioritization score further based on the change over the time period in the risk score for the patient.

12. The method of claim 7, further comprising:
determining a measure representing a willingness of the patient to receive care; and
determining the patient prioritization score further based on the measure representing the willingness of the patient to receive care.

13. The method of claim 7, further comprising:
determining a prediction of future costs associated with care of the patient; and
determining the patient prioritization score further based on the prediction of the future costs associated with care of the patient.

14. The method of claim 7, wherein receiving the healthcare data associated with the patient comprises:
using a large language model to generate a prompt requesting input from the patient; and
receiving at least some of the healthcare data in response to generating the prompt.

15. At least one non-transitory computer-readable storage medium having instructions encoded thereon that, when executed by at least one computer processor, cause the at least one computer processor to perform a method comprising:
generating a graphical user interface (GUI) including a prompt to a healthcare professional to administer a level of care to a patient; and
providing the GUI to a display,
wherein the prompt, included in the GUI, to the healthcare professional to administer the level of care to the patient is determined based on a patient prioritization score of the patient, the patient prioritization score of the patient being determined based on a care gap score for the patient representing opportunities to improve care of the patient and a risk score for the patient representing risks to health of the patient.

16. The at least one non-transitory computer-readable storage medium of claim 15, wherein the method further comprises:
receiving healthcare data associated with the patient;
determining, based on the healthcare data for the patient, the care gap score for the patient representing opportunities to improve care of the patient;
determining, based on the healthcare data for the patient, the risk score for the patient representing risks to health of the patient; and
determining, based on the care gap score and the risk score, the patient prioritization score of the patient.

17. The at least one non-transitory computer-readable storage medium of claim 16, wherein determining a care gap score for the patient based on the healthcare data for the patient comprises:
applying a rule to the healthcare data for the patient, wherein the rule is associated with a care gap;
determining, based on a result of applying the rule, whether the patient has the care gap associated with the rule;
providing an individual score for the care gap based on a result of determining whether the patient has the care gap;
applying a weight to the individual score for the care gap; and
determining the care gap score based on the weight and the individual score.

18. The at least one non-transitory computer-readable storage medium of claim 16, wherein the method further comprises:
determining a change over a time period in the risk score for the patient; and
determining the patient prioritization score further based on the change over the time period in the risk score for the patient.

19. The at least one non-transitory computer-readable storage medium of claim 16, wherein the method further comprises:
determining a measure representing a willingness of the patient to receive care; and
determining the patient prioritization score further based on the measure representing the willingness of the patient to receive care.

20. The at least one non-transitory computer-readable storage medium of claim 16, wherein the method further comprises:
determining a prediction of future costs associated with care of the patient; and
determining the patient prioritization score further based on the prediction of the future costs associated with care of the patient.
